SEQUENCE LISTING (7 sequences)

SEQ ID NO: 1 - protein, 269 aa, Humicola lanuginosa [sequence omitted]

SEQ ID NO: 2 - protein, 269 aa, synthetic construct
DVSADLLNQFKLFAQYSAAAYCGRNNDAPAGTNITCSANACPLVEAADATFLYSFENSGVGDVTGFLAVDNTNKLIVLSFRGSRSIENWITNLNFDLVDINDICSGCRGHDGFTSSWRSVADTLRQAVDDAVAAHPDYKVVVTGHSLGGALATLAGADLRNNGYDVDLYTYGAPRVGNRAFAEFITVQTGGTLYRVTHTNDVVPRLPPRQFGYSHPSPEYWIKSGTLVPVRRRDIVKIEGIDATGGNNQNNTPDIPAHLWYFGLIGTCI

SEQ ID NO: 3 - protein, 544 aa, Geotrichum candidum
QAPTAVLNGNEVISGVLEGKVDTFKGIPFADPPVGDLRFKHPQPFTGSYQGLKANDFSSACMQLDPGNAISLLDKVVGLGKIIPDNLRGPLYDMAQGSVSMNEDCLYLNVFRPAGTKPDAKLPVMVWIYGGAFVFGSSASYPGNGYVKESVEMGQPVVFVSINYRTGPYGFLGGDAITAEGNTNAGLHDQRKGLEWVSDNIANFGGDPDKVMIFGESAGAMSVAHQLVAYGGDNTYNGKQLFHSAILQSGGPLPYFDSTSVGPESAYSRFAQYAGCDASAGDNETLACLRSKSSDVLHSAQNSYDLKDLFGLLPQFLGFGPRPDGNIIPDAAYELYRSGRYAKVPYITGNQEDEGTILAPVAINATTTPHVKKWLKYICSEASDASLDRVLSLYPGSWSEGAPFRTGILNALTPQFKRIAAIFTDLLFQSPRRVMLNATKDVNRWTYLATQLHNLVPFLGTFHGSDLLFQYYVDLGPSSAYRRYFISFANHHDPNVGTNLKQWDMYTDAGREMLQIHMIGNSMRTDDFRIEGISNFESDVTLFG

SEQ ID NO: 4 - protein, 544 aa, Geotrichum candidum
QAPTAVLNGNEVISGVLEGKVDTFKGIPFADPPVGDLRFKHPQPFTGSYQGLKANDFSSACMQLDPGNAISLLDKVVGLGKIIPDNLRGPLYDMAQGSVSMNEDCLYLNVFRPAGTKPDAKLPVMVWIYGGAFVFGSSASYPGNGYVKESVEMGQPVVFVSINYRTGPYGFLGGDAITAEGNTNAGLHDQRKGLEWVSDNIANFGGDPDKVMIFGESAGAMSVAHQLVAYGGDNTYNGKQLFHSAILQSGGPLPYFDSTSVGPESAYSRFAQYAGCDASAGDNETLACLRSKSSDVLHSAQNSYDLKDLFGLLPQFLGFGPRPDGNIIPDAAYELYRSGRYAKVPYITGNQEDEGTILAPVAINATTTPHVKKWLKYICSEASDASLDRVLSLYPGSWSEGAPFRTGILNALTPQFKRIAAIFTDLLFQSPRRVMLNATKDVNRWTYLATQLHNLVPFLGTFHGSDLLFQYYVDLGPSSAYRRYFISFANHHDPNVGTNLKQWDMYTDSGKEMLQIHMIGNSMRTDDFRIEGISNFESDVTLFG

SEQ ID NO: 5 - protein, 544 aa, Geotrichum candidum
QAPTAVLNGNEVISGVLEGKVDTFKGIPFADPPVGDLRFKHPQPFTGSYQGLKANDFSSACMQLDPGNAISLLDKVVGLGKIIPDNLRGPLYDMAQGSVSMNEDCLYLNVFRPAGTKPDAKLPVMVWIYGGAFVFGSSASYPGNGYVKESVEMGQPVVFVSINYRTGPYGFLGGDAITAEGNTNAGLHDQRKGLEWVSDNIANFGGDPDKVMIFGESAGAMSVAHQLVAYGGDNTYNGKQLFHSAILQSGGPLPYFDSTSVGPESAYSRFAQYAGCDASAGDNETLACLRSKSSDVLHSAQNSYDLKDLFGLLPQFLGFGPRPDGNIIPDAAYELYRSGRYAKVPYITGNQEDEGTILAPVAINATTTPHVKKWLKYICSEASDASLDRVLSLYPGSWSEGAPFRTGILNALTPQFKRIAAIFTDLLFQSPRRVMLNATKDVNRWTYLATQLHNLVPFLGTFHGSDLLFQYYVDLGPSSAYRRYFISFANHHDPNVGTNLKQWDMYTDSGKEMLQIHMIGNSMRTDDFRIEGISNFETDVNLYG

SEQ ID NO: 6 - protein, 544 aa, Geotrichum candidum
QAPTAVLNGNEVISGVLEGKVDTFKGIPFADPPVGDLRFKHPQPFTGSYQGLKANDFSSACMQLDPGNAISLLDKVVGLGKIIPDNLRGPLYDMAQGSVSMNEDCLYLNVFRPAGTKPDAKLPVMVWIYGGAFVFGSSASYPGNGYVKESVEMGQPVVFVSINYRTGPYGFLGGDAITAEGNTNAGLHDQRKGLEWVSDNIANFGGDPDKVMIFGESAGAMSVAHQLVAYGGDNTYNGKQLFHSAILQSGGPLPYFDSTSVGPESAYSRFAQYAGCDASAGDNETLACLRSKSSDVLHSAQNSYDLKDLFGLLPQFLGFGPRPDGNIIPDAAYELYRSGRYAKVPYITGNQEDEGTILAPVAINATTTPHVKKWLKYICSEASDASLDRVLSLYPGSWSEGAPFRTGILNALTPQFKRIAAIFTDLLFQSPRRVMLNATKDVNRWTYLATQLHNLVPFLGTFHGSDLLFQYYVDLGPSSAYRRYFISFANHHDPNVGTNLKQWDMYTDSGKEMLQIHMIGNSMRTDDFRIEGISNFESDVNLYG

SEQ ID NO: 7 - protein, 544 aa, Geotrichum candidum
QAPTAVLNGNEVISGVLEGKVDTFKGIPFADPPVGDLRFKHPQPFTGSYQGLKANDFSSACMQLDPGNAFSLLDKVVGLGKILPDNLRGPLYDMAQGSVSMNEDCLYLNVFRPAGTKPDAKLPVMVWIYGGAFVFGSSASYPGNGYVKESVEMGQPVVFVSINYRTGPYGFLGGDAITAEGNTNAGLHDQRKGLEWVSDNIANFGGDPDKVMIFGESAGAMSVAHQLVAYGGDNTYNGKQLFHSAILQSGGPLPYFDSTSVGPESAYSRFAQYAGCDTSASDNDTLACLRSKSSDVLHSAQNSYDLKDLFGLLPQFLGFGPRPDGNIIPDAAYELYRSGRYAKVPYITGNQEDEGTILAPVAINATTTPHVKKWLKYICSQASDASLDRVLSLYPGSWSEGSPFRTGILNALTPQFKRIAAIFTDLLFQSPRRVMLNATKDVNRWTYLATQLHNLVPFLGTFHGSDLLFQYYVDLGPSSAYRRYFISFANHHDPNVGTNLQQWDMYTDAGKEMLQIHMIGNSMRTDDFRIEGISNFESDVTLFG

(19) United States
(12) Patent Application Publication
(10) Pub. No.: US 2026/0055348 A1
(43) Pub. Date: Feb. 26, 2026

(54) Hard Surface Cleaning Composition and Cleaning Method

(71) Applicant: Novozymes A/S, Bagsvaerd (DK)

(72) Inventors: Weixiao CHEN, Beijing (CN); Yang LIU, Beijing (CN)

(73) Assignee: Novozymes A/S, Bagsvaerd (DK)

(21) Appl. No.: 19/100,142

(22) PCT Filed: Aug. 4, 2023
(86) PCT No.: PCT/CN2023/111270

§ 371 Date: Jan. 31, 2025

(30) Foreign Application Priority Data
Aug. 4, 2022 (WO) PCT/CN2022/110270
Nov. 18, 2022 (WO) PCT/CN2022/132844

(51) Int. Cl. C11D 3/386; C11D 3/20; C11D 3/38; C11D 3/43

(52) U.S. Cl. CPC C11D 3/38627; C11D 3/2068; C11D 3/381; C11D 3/43; C12Y 301/01003

(57) ABSTRACT

A hard surface cleaning composition comprising: (a) 0.1 to 60 wt % of a surfactant system; (b) 0.1 to 40 wt % of a solvent, wherein the solvent is selected from the group consisting of a lower alkanol, a benzyl alcohol, a lower alkyl ether, a glycol, an aryl glycol ether, a lower alkyl glycol ether, a glycerol ketal, an ester, a hydrocarbon/ester blend, a dibasic ester, a linear alcohol ethoxylate, a branched alcohol, oxo alcohol ethoxylate, and combinations thereof; and (c) optionally 0.001 to 10 wt % of a lipase.

### REFERENCE TO A SEQUENCE LISTING

This application contains a Sequence Listing in computer readable form, which is incorporated herein by reference.

### FIELD OF THE INVENTION

The present invention relates to a cleaning composition for removing stains, particularly lipid or greasy soils, from a hard surface e.g. a surface of dishware or kitchen bench. The invention also relates to methods for cleaning hard surface as well as ready-to-use items comprising said composition.

### BACKGROUND OF INVENTION

Hard surface cleaning composition is useful on a variety of surfaces, such as industrial surfaces and household surfaces (e.g., kitchen surfaces). During use between cleanings, build-up of varied soils, such as greasy soils, dirt and even biofilms, occurs on these surfaces quickly and soon becomes hard to be removed. To ease cleaning, conventional hard surface cleaners are made to have a high pH (normally above pH 10). However, high pH cleaners are harsh to hand skin and to environment as well. High pH may also cause damage to the surface materials e.g., coatings, and result in rough surfaces which in return make soil removal even more difficult. In addition, the wash performance of high pH conventional detergents might not be good enough for tough stains such as kitchen oily stains in some cases.

Accordingly, there is a need to develop a hard surface cleaning composition that has a milder pH with improved wash performance compared to that of the high pH conventional cleaning composition.

On the other hand, lipase is not show satisfying wash performance boosting in some current detergent formulations, which limits the application of lipase in e.g., hard surface cleaning detergent. It is therefore necessary to develop a detergent formulation that enables lipase to perform significantly and consistently so as to facilitate greasy soils e.g., kitchen oily stains removal.

### SUMMARY OF THE INVENTION

The present invention relates to a hard surface cleaning composition comprising:
(a) 0.1 to 60 wt % of a surfactant system;
(b) 0.1 to 40 wt % of a solvent, wherein the solvent is selected from the group consisting of a lower alkanol, a benzyl alcohol, a lower alkyl ether, a glycol, an aryl glycol ether, a lower alkyl glycol ether, a glycerol ketal, an ester, a hydrocarbon/ester blend, a dibasic ester, alcohol ethoxylate such as a linear alcohol ethoxylate, a branched alcohol or oxo alcohol ethoxylate, and combinations thereof; and optionally
(c) 0.001 to 10 wt % of a lipase.

The cleaning composition of the invention may further comprise one or more components selected from the group of builders, chelating agents, dispersants, enzyme stabilizers, catalytic materials, bleach activators, hydrogen peroxide, sources of hydrogen peroxide, preformed peracids, polymeric dispersing agents, suds suppressors, dyes, perfumes, structure elasticizing agents, carriers, hydrotropes, processing aids, pigments and/or additional enzymes.

In a preferred embodiment a cleaning composition of the invention comprises one or more of the lipases shown in any one of SEQ ID NOs: 1, 2, 3, 4, 5, 6 or 7, or variants thereof.

In an embodiment the cleaning composition of the invention comprises a commercial lipase product selected from the group of Lipolase™, Lipex™; Lipolex™, Lipoclean™, Lipex Evity 100L, Lipex Evity 105T, Lipex Evity 200L (from Novozymes), Lumafast (originally from Genencor), Preferenz L100 (Danisco US Inc.), and Lipomax (originally from Gist-Brocades).

In an embodiment the cleaning composition of the invention comprises a solvent selected from ethanol, propanol, isopropanol and butanol, isobutanol, ethylene glycol, diethylene glycol, triethylene glycol, propylene glycol, dipropylene glycol, mixed ethylene-propylene glycol ethers, ethylene glycol phenyl ether, propylene glycol phenyl ether, propylene glycol methyl ether, propylene glycol propyl ether, dipropylene glycol methyl ether, tripropylene glycol butyl ether, tripropylene glycol methyl ether, ethylene glycol butyl ether, diethylene glycol methyl ether, diethylene glycol butyl ether, ethylene glycol dimethyl ether, ethylene glycol propyl ether, diethylene glycol ethyl ether, triethylene glycol methyl ether, triethylene glycol ethyl ether, triethylene glycol butyl ether, ethylene glycol monobutyl ether, diethylene glycol monobutyl ether, triethylene glycol monobutyl ether, dipropylene glycol mono butyl ether, diethylene glycol monobutyl ether, hexyl ethoxylate (e.g., ethylene glycol monohexyl ether), 2-ethoxyethylalcohol, 2-(2-ethoxyethoxy)ethanol, triethylene glycol monoethyl ether, 2-(hexyloxy)ethanol, 2-(2-hexyloxyethoxy)ethanol, phenoxyethanol, and combination thereof, e.g. a combination of ethanol and diethylene glycol monobutyl ether, glycol ether, propylene glycol or dipropylene glycol.

The invention also relates to methods for cleaning a hard surface, comprising contacting the surface with a hard surface cleaning composition of the invention.

The invention further relates to a ready-to-use item (e.g., a wipe or a sponge) for cleaning a hard surface, wherein the item comprises a hard surface cleaning composition of the invention.

### OVERVIEW OF SEQUENCES

SEQ ID NO: 1 is the wild-type Thermomyces lanuginosus lipase (synonym Humicola lanuginosa DSM 4109 lipase) often referred to simply as “TLL”.
SEQ ID NO: 2 is a variant of the Thermomyces lanuginosus lipase disclosed as SEQ ID NO: 2 in WO 2019/154952.
SEQ ID NO: 3 is a lipase from Geotrichum candidum.
SEQ ID NO: 4 is a lipase from Geotrichum candidum.
SEQ ID NO: 5 is a lipase from Geotrichum candidum.
SEQ ID NO: 6 is a lipase from Geotrichum candidum.
SEQ ID NO: 7 is a lipase from Geotrichum candidum.

### BRIEF DESCRIPTION OF THE DRAWINGS

The present invention will now be described in more detail, with reference to the appended drawings showing an example embodiment of the invention, wherein:

FIG. 1 shows the washed DM-90 stain tiles under different wash conditions.

FIG. 2 shows the wash performance of Model detergent V2 (pH8) with or without lipase on mixed grease stains, with 15 min soaking time.

### DEFINITIONS

As used herein, the singular forms “a”, “an”, and “the” are intended to include the plural forms as well, unless the context clearly indicates otherwise.

If not indicated otherwise, all references to percentages in relation to the disclosed compositions relate to wt % relative to the total weight of the respective composition.

Lipase: The terms “lipase”, “lipase enzyme”, “lipolytic enzyme”, “lipid esterase”, “lipolytic polypeptide”, and “lipolytic protein” refers to an enzyme in class EC3.1.1 as defined by Enzyme Nomenclature. It may have lipase activity (triacylglycerol lipase, EC3.1.1.3), cutinase activity (EC3.1.1.74), sterol esterase activity (EC3.1.1.13) and/or wax-ester hydrolase activity (EC3.1.1.50). For purposes of the present invention lipase activity (i.e. the hydrolytic activity of the lipase) may be determined with a pNP assay using substrates with various chain length as described in Assay I in the Example-section.

Fragment: The term “fragment” means a polypeptide having one or more (e.g., several) amino acids absent from the amino and/or carboxyl terminus of a polypeptide; wherein the fragment has lipase activity. In one aspect, a fragment contains at least 50%, at least 55%, at least 60%, at least 65%, at least 70%, at least 75%, at least 80%, at least 85%, at least 90%, or at least 95% but less than 100% of the number of amino acids 1 to 269 of SEQ ID NO: 1 or 2.

Parent or parent lipase: The term “parent” or “parent lipase” means a lipase to which an alteration is made to produce the enzyme variants. The parent lipase may be a naturally occurring (wild-type) polypeptide but may also be a variant and/or fragment thereof. In preferred embodiments, the parent lipase may be the ones shown in SEQ ID NOs: 1, 2, 3, 4, 5, 6 or 7.

Wild-type lipase: The term “wild-type” lipase means a lipase expressed by a naturally occurring microorganism, such as a bacterium, yeast, or filamentous fungus found in nature. In an embodiment, the wild-type lipase may be the one shown in SEQ ID NO: 1, which is derived from Thermomyces lanuginosus DSM 4109 (synonym Humicola lanuginosa DSM 4109) or the Absidia sp. lipase shown in SEQ ID NO: 3 of WO 2021/001400.

Cleaning components: The term “cleaning components” is defined herein to mean the types of chemicals which can be used in cleaning compositions. Examples of cleaning components are alkalis, surfactants, solvents, hydrotropes, builders, co-builders, chelators or chelating agents, bleaching system or bleach components, polymers, foam boosters, suds suppressors, dispersants, dye transfer inhibitors, perfume, bactericides, fungicides, corrosion inhibitors, soil suspending agents, soil release polymers, antiredeposition agents, enzyme inhibitors or stabilizers, enzyme activators, antioxidants, preservatives, solvents and solubilizers.

Cleaning composition: The term “cleaning composition (may also be referred as “detergent composition”) refers to compositions that find use in the removal of undesired compounds from items to be cleaned, such as a hard surface). The cleaning composition may be used to e.g., for household cleaning and industrial cleaning. The terms encompass any materials/compounds selected for the particular type of cleaning composition desired and the form of the product (e.g., liquid, gel, powder, granulate, paste, or spray compositions). In addition to containing a lipase of the invention, the cleaning composition of the present invention may contain one or more additional enzymes (such as amylases, protease, cellulases, mannanases, hemicellulases, peroxidases, xylanases, phospholipases, esterases, cutinases, keratinases, reductases, oxidases, phenoloxidases, lipoxygenases, ligninases, pullulanases, malanases, ß-glucanases, arabinosidases, hyaluronidase, laccase, deoxyribonucleases (DNases), hexosaminidases and peroxidases, or any mixture thereof), and/or cleaning components as described above.

Alkyl: The term “alkyl” refers to a straight or branched chain monovalent hydrocarbon radical having a specified number of carbon atoms. Alkyl groups may be unsubstituted or substituted with substituents that do not interfere with the specified function of the composition and may be substituted once or twice with the same or different group. Substituents may include alkoxy, hydroxy, mercapto, amino, alkyl substituted amino, nitro, carboxy, carbanyl, carbanyloxy, cyano, methylsulfonylamino, or halogen, for example. Examples of “alkyl” include, but are not limited to, methyl, ethyl, n-propyl, isopropyl, n-butyl, s-butyl, t-butyl, n-pentyl, n-hexyl, 3-methylpentyl, and the like.

Sequence identity: The relatedness between two amino acid sequences or between two nucleotide sequences is described by the parameter “sequence identity”. For purposes of the present invention, the sequence identity between two amino acid sequences is determined as the output of “longest identity” using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the nobrief option must be specified in the command line. The output of Needle labeled “longest identity” is calculated as follows:

$$( Identical Residues \times 100 ) / ( Length of Alignment - Total Number of Gaps in Alignment )$$

Variant: The term “variant” means a polypeptide/enzyme having similar activity as the parent enzyme comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position. In the context of the present invention, a variant of an identified lipase has the enzymatic activity of the parent. In one embodiment, the lipase activity of the variant is increased with reference to the parent lipase, e.g. the enzyme comprising or consisting of the amino acid sequence of SEQ ID NO: 1 or the enzyme comprising or consisting of the amino acid sequence set forth in SEQ ID NO: 2.

Hard surface: The term “hard surface” includes household surfaces e.g., showers, sinks, toilets, bathtubs, countertops, windows, mirrors, floors, and the like, and industrial surfaces such as transportation vehicles, storage tanks, bioreactors, fermenters, mix vessels, pipelines, bottling line equipment and other equipment used in biotech manufacturing, e.g., pharmaceutical manufacturing, food and beverage manufacturing. The term may also include surfaces of medical or dental devices which comes into contact with a patient, where the patient may be a human or an animal. Exemplary hard surface may be a surface of a steel, a rubber, a plastic, a glass, a ceramic, melamine, wood, coated surfaces, cement countertops, kitchen countertops, endoscopes, arthoscopes scalpels, hemostats, Kocher forceps and tracheotomes and the like.

### Conventions for Designation of Variants

For purposes of the present invention, the lipase disclosed as SEQ ID NO: 1 is used to determine the corresponding amino acid residue in another lipase. The amino acid sequence of another lipase is aligned with SEQ ID NO: 1, and based on the alignment, the amino acid position number corresponding to any amino acid residue in the polypeptide disclosed in SEQ ID NO: 1 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

Identification of the corresponding amino acid residue in another lipase can be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

When the other enzyme has diverged from the lipase of SEQ ID NO: 1 such that traditional sequence-based comparison fails to detect their relationship (Lindahl and Elofsson, 2000, J. Mol. Biol. 295: 613-615), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example, the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homologs (e.g., Holm and Park, 2000, Bioinformatics 16: 566-567).

In describing variants used in context of the invention, the nomenclature described below is adapted for ease of reference. The accepted IUPAC single letter or three letter amino acid abbreviation is employed.

Substitutions: For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine (R) and serine (S) with phenylalanine (F), respectively.

Deletions: For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”.

Insertions: For an amino acid insertion, the following nomenclature is used: Original amino acid, position, original amino acid, inserted amino acid. Accordingly, the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G - K - A |

Multiple alterations. Variants comprising multiple alterations are separated by addition marks (+”), e.g., “Arg170Tyr+Gly195Glu” or “R170Y+G195E” representing a substitution of arginine and glycine at positions 170 and 195 with tyrosine and glutamic acid, respectively.

Different alterations. Where different alterations can be introduced at a position, the different alterations are separated by a comma, e.g., “Arg170Tyr, Glu” or “R170Y, E” represents a substitution of arginine at position 170 with tyrosine or glutamic acid. Thus, “Tyr167Gly, Ala+Arg170Gly, Ala” designates the following variants:
“Tyr167Gly+Arg170Gly”, “Tyr167Gly+Arg170Ala”, “Tyr167Ala+Arg170Gly”, and “Tyr167Ala+Arg170Ala”.

### DETAILED DESCRIPTION OF THE INVENTION

The inventors have surprisingly found that, inclusion of a lipase together with a solvent into a hard surface cleaning composition provides an enhanced cleaning effect as well as an improved wash efficiency, with regard to especially oily or greasy stain removal. Consequently, compared to the high pH conventional hard surface cleaning detergent, the cleaning composition of the present invention can not only provide a better cleaning effect, but also can save time and labor cost. In addition, the pH of the cleaning composition can be lowered from e.g. pH 11 to milder pH (e.g., below pH 9 or a neutral pH), without compromising the cleaning effect/wash performance.

### Cleaning Compositions of the Invention

The first aspect of the present invention relates to a hard surface cleaning composition comprising:
(a) 0.1 to 60 wt % of a surfactant system;
(b) 0.1 to 40 wt % of a solvent, wherein the solvent is selected from the group consisting of a lower alkanol, a benzyl alcohol, a lower alkyl ether, a glycol, an aryl glycol ether, a lower alkyl glycol ether, a glycerol ketal, an ester, a hydrocarbon/ester blend, a dibasic ester, alcohol ethoxylate such as a linear alcohol ethoxylate, a branched alcohol or oxo alcohol ethoxylate, and combinations thereof; and optionally
(c) 0.001 to 10 wt % of a lipase.

The composition of the present invention may be formulated as a ready-to-use detergent or as a concentrate (e.g., 2×, 5× or 10× concentrated) detergent that can be diluted onsite before application. Enzyme(s) (e.g., a lipase) may be added when formulating the concentrate or added to the diluted version onsite.

### Composition Components

The non-limiting list of composition components illustrated hereinafter are suitable for use in the compositions and methods of the invention and may be desirably incorporated in certain embodiments of the invention, e.g. to assist or enhance cleaning performance, for treatment of the hard surface to be cleaned, or to modify the aesthetics of the composition as is the case with perfumes, colorants, dyes or the like. The precise nature of these components, and levels of incorporation thereof, will depend on the physical form of the composition and the nature of the cleaning operation for which it is to be used. Although components mentioned below are categorized by general header according to a particular functionality, this is not to be construed as a limitation, as a component may comprise additional functionalities as will be appreciated by the skilled artisan.

Suitable component materials include, but are not limited to, surfactants, builders, chelating agents, dispersants, enzymes, and enzyme stabilizers, catalytic materials, bleach activators, hydrogen peroxide, sources of hydrogen peroxide, preformed peracids, polymeric dispersing agents, clay soil removal, suds suppressors, dyes, perfumes, perfume delivery systems, carriers, hydrotropes, processing aids and solvents. In addition to the disclosure below, suitable examples of such other components and levels of use are found in U.S. Pat. Nos. 5,576,282, 6,306,812, and 6,326,348 hereby incorporated by reference.

### Surfactants or Surfactant Systems

A detergent or cleaning composition of the present invention comprises a surfactant or surfactant system, a lipase and a solvent compound. In an embodiment, the surfactant system comprises at least one surfactant selected from nonionic surfactants, anionic surfactants, cationic surfactants, ampholytic surfactants, zwitterionic surfactants, semi-polar nonionic surfactants, and mixtures thereof.

The surfactant(s) is(are) typically present at a level of from 0.1 to 60 wt %, from 0.2 to 40 wt %, from 0.5 to 30 wt %, from 1 to 50 wt %, from 1 to 40 wt %, from 1 to 30 wt %, from 1 to 20 wt %, from 3 to 10 wt %, from 3 to 5 wt %, from 5 to 40 wt %, from 5 to 30 wt %, from 5 to 15 wt %, from 2 to 20 wt %, from 3 to 6 wt %, from 8 to 12 wt %, from 10 to 12 wt %, from 20 to 25 wt % or from 25-60%.

Suitable anionic surfactants include sulphate and sulphonate detersive surfactants.

Suitable sulphonate surfactants include alkyl benzene sulphonate, in one aspect, C₁₀₋₁₃ alkyl benzene sulphonate. Suitable alkyl benzene sulphonate (LAS) may be obtained, by sulphonating commercially available linear alkyl benzene (LAB); suitable LAB includes low 2-phenyl LAB, such as Isochem® or Petrelab®, other suitable LAB include high 2-phenyl LAB, such as Hyblene®. A suitable anionic detersive surfactant is alkyl benzene sulphonate that is obtained by DETAL catalyzed process, although other synthesis routes, such as HF, may also be suitable. In one aspect a magnesium salt of LAS is used.

Suitable sulphate surfactants include alkyl sulphate, in one aspect, C₈₋₁₈ alkyl sulphate, or predominantly C₁₂ alkyl sulphate.

Another suitable sulphate surfactant is alkyl alkoxylated sulphate, in one aspect, alkyl ethoxylated sulphate, in one aspect, a C₈₋₁₈ alkyl alkoxylated sulphate, in another aspect, a C₈₋₁₈ alkyl ethoxylated sulphate, typically the alkyl alkoxylated sulphate has an average degree of alkoxylation of from 0.5 to 20, or from 0.5 to 10, typically the alkyl alkoxylated sulphate is a C₈₋₁₈ alkyl ethoxylated sulphate having an average degree of ethoxylation of from 0.5 to 10, from 0.5 to 7, from 0.5 to 5 or from 0.5 to 3.

The alkyl sulphate, alkyl alkoxylated sulphate and alkyl benzene sulphonates may be linear or branched, substituted or un-substituted.

The surfactant may be a mid-chain branched detersive surfactant, in one aspect, a mid-chain branched anionic detersive surfactant, in one aspect, a mid-chain branched alkyl sulphate and/or a mid-chain branched alkyl benzene sulphonate, e.g. a mid-chain branched alkyl sulphate. In one aspect, the mid-chain branches are C₁₋₄ alkyl groups, typically methyl and/or ethyl groups.

Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS) or sodium lauryl sulfate (SLS), fatty alcohol sulfates (FAS), primary alcohol sulfates (PAS), alcohol ethersulfates (AES or AEOS or FES, also known as alcohol ethoxysulfates or fatty alcohol ether sulfates), sodium laureth sulfate (SLES), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or soap, and combinations thereof.

Anionic surfactant may be present at a level of from 0.2 to 30 wt %, from 0.5 to 25 wt %, from 1 to 20 wt %, from 2 to 15 wt %, from 3 to 12 wt %, from 4 to 10 wt %, from 5 to 15 wt % or from 6 to 8 wt %.

Suitable nonionic surfactants are selected from the group consisting of: C₈-C₁₈ alkyl ethoxylates, such as, NEODOL®; C₆-C₁₂ alkyl phenol alkoxylates wherein the alkoxylate units may be ethyleneoxy units, propyleneoxy units or a mixture thereof; C₁₂-C₁₈ alcohol and C₆-C₁₂ alkyl phenol condensates with ethylene oxide/propylene oxide block polymers such as Pluronic®; C₁₄-C₂₂ mid-chain branched alcohols; C₁₄-C₂₂ mid-chain branched alkyl alkoxylates, typically having an average degree of alkoxylation of from 1 to 30; alkylpolysaccharides, in one aspect, alkylpolyglycosides; polyhydroxy fatty acid amides; ether capped poly(oxyalkylated) alcohol surfactants; and mixtures thereof.

Suitable nonionic detersive surfactants include alkyl polyglucoside and/or an alkyl alkoxylated alcohol.

In one aspect, nonionic detersive surfactants include alkyl alkoxylated alcohols, in one aspect C₈₋₁₈ alkyl alkoxylated alcohol, e.g. a C₈₋₁₈ alkyl ethoxylated alcohol, the alkyl alkoxylated alcohol may have an average degree of alkoxylation of from 1 to 50, from 1 to 30, from 1 to 20, or from 1 to 10. In one aspect, the alkyl alkoxylated alcohol may be a C₈₋₁₈ alkyl ethoxylated alcohol having an average degree of ethoxylation of from 1 to 10, from 1 to 7, more from 1 to 5 or from 3 to 7. The alkyl alkoxylated alcohol can be linear or branched and substituted or un-substituted. Suitable nonionic surfactants include Lutensol®.

Non-limiting examples of nonionic surfactants include alcohol ethoxylates (AE or AEO), such as linear alcohol (C12-15) ethoxylate (LAE), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof.

Suitable cationic detersive surfactants include alkyl pyridinium compounds, alkyl quaternary ammonium compounds, alkyl quaternary phosphonium compounds, alkyl ternary sulphonium compounds, and mixtures thereof.

Suitable cationic detersive surfactants are quaternary ammonium compounds having the general formula: (R)(R₁)(R₂)(R₃)N⁺X⁻, wherein, R is a linear or branched, substituted or unsubstituted C₆₋₁₈ alkyl or alkenyl moiety, R₁ and R₂ are independently selected from methyl or ethyl moieties, R₃ is a hydroxyl, hydroxymethyl or a hydroxyethyl moiety, X is an anion which provides charge neutrality, suitable anions include: halides, e.g. chloride; sulphate; and sulphonate. Suitable cationic detersive surfactants are mono-C₆₋₁₈ alkyl mono-hydroxyethyl di-methyl quaternary ammonium chlorides. Highly suitable cationic detersive surfactants are mono-C₈₋₁₀ alkyl mono-hydroxyethyl di-methyl quaternary ammonium chloride, mono-C₁₀₋₁₂ alkyl mono-hydroxyethyl di-methyl quaternary ammonium chloride and mono-C₁₀ alkyl mono-hydroxyethyl di-methyl quaternary ammonium chloride.

Non-limiting examples of cationic surfactants include alkyldimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, ester quats, and combinations thereof.

Suitable amphoteric/zwitterionic surfactants include amine oxides and betaines such as alkyldimethylbetaines, sulfobetaines, or combinations thereof. Amine-neutralized anionic surfactants—Anionic surfactants and adjunct anionic cosurfactants, may exist in an acid form, and said acid form may be neutralized to form a surfactant salt which is desirable for use in the present detergent compositions. Typical agents for neutralization include the metal counterion base such as hydroxides, eg, NaOH or KOH. Further preferred agents for neutralizing anionic surfactants of the present invention and adjunct anionic surfactants or cosurfactants in their acid forms include ammonia, amines, or alkanolamines. Alkanolamines are preferred. Suitable non-limiting examples including monoethanolamine, diethanolamine, triethanolamine, and other linear or branched alkanolamines known in the art; e.g., highly preferred alkanolamines include 2-amino-1-propanol, 1-aminopropanol, monoisopropanolamine, or 1-amino-3-propanol. Amine neutralization may be done to a full or partial extent, e.g. part of the anionic surfactant mix may be neutralized with sodium or potassium and part of the anionic surfactant mix may be neutralized with amines or alkanolamines.

Non-limiting examples of semipolar surfactants include amine oxides (AO) such as alkyldimethylamineoxide

The surfactant system of the present invention may comprise at least one surfactant selected from the group consisting of nonionic surfactants, anionic surfactants, cationic surfactants, ampholytic surfactants, zwitterionic surfactants, semi-polar nonionic surfactants, and mixtures thereof. Preferred weight ratios of anionic to nonionic surfactant are at least 2:1, or at least 1:1 to 1:10.

In an embodiment, the composition of the invention comprises one or more anionic surfactant and/or one or more nonionic surfactant.

In a preferred embodiment, the composition of the invention comprises one or more anionic surfactants, preferably from the group of linear alkylbenzenesulfonic acid (LAS), alcohol ethersulfate (AEOS) and/or alkyl sulfate (AS), in particular sodium lauryl sulfate (SLS) and sodium laureth sulfate (SLES).

In an embodiment, the composition of the invention comprises one or more nonionic surfactants, preferably alcohol ethoxylate (AEO), in particular linear alcohol (C₁₂₋₁₅) ethoxylate (LAE).

In one embodiment, the composition of the invention comprises the anionic surfactant linear alkylbenzenesulfonic acid (LAS) and the nonionic surfactant alcohol ethoxylate (AEO).

In one embodiment, the composition of the invention comprises the anionic surfactant sodium laureth sulfate (SLES) and the nonionic surfactant alcohol ethoxylate (AEO).

Nonionic surfactant may be present at a level of from 0.2 to 30 wt %, from 0.5 to 25 wt %, from 1 to 20 wt %, from 2 to 15 wt %, from 3 to 12 wt %, from 4 to 10 wt %, or from 6 to 8 wt %.

In an embodiment the ratio between anionic surfactant and nonionic surfactant is in the range 1:10 to 10:1, such as 1:5 to 5:1, or 1:3 to 2:1.

In a specific embodiment, the composition comprises the surfactant system as described in table 1 below.

### Solvent

The cleaning compositions of the invention may include a solvent to e.g. improve soil removal property or to adjust viscosity of the final composition. Suitable solvents useful in the present invention include, but are not limited to: a lower alkanol, a benzyl alcohol, a lower alkyl ether, a glycol, an aryl glycol ether, a lower alkyl glycol ether, a glycerol ketal, an ester, a hydrocarbon/ester blend, a dibasic ester, alcohol ethoxylate such as a linear alcohol ethoxylate, a branched alcohol or oxo alcohol ethoxylate, and the like.

Exemplary solvents include, but are not limited to: ethanol, propanol, isopropanol and butanol, isobutanol, ethylene glycol, diethylene glycol, triethylene glycol, propylene glycol, dipropylene glycol, mixed ethylene-propylene glycol ethers, ethylene glycol phenyl ether, propylene glycol phenyl ether, propylene glycol methyl ether, propylene glycol propyl ether, dipropylene glycol methyl ether, tripropylene glycol butyl ether, tripropylene glycol methyl ether, ethylene glycol butyl ether, diethylene glycol methyl ether, diethylene glycol butyl ether, ethylene glycol dimethyl ether, ethylene glycol propyl ether, diethylene glycol ethyl ether, triethylene glycol methyl ether, triethylene glycol ethyl ether, triethylene glycol butyl ether, ethylene glycol monobutyl ether, diethylene glycol monobutyl ether, triethylene glycol monobutyl ether, dipropylene glycol mono butyl ether, diethylene glycol monobutyl ether, hexyl ethoxylate (e.g., ethylene glycol monohexyl ether), 2-ethoxyethylalcohol, 2-(2-ethoxyethoxy)ethanol, triethylene glycol monoethyl ether, 2-(hexyloxy)ethanol, 2-(2-hexyloxyethoxy)ethanol, phenoxyethanol, and combination thereof, e.g. a combination of ethanol and diethylene glycol monobutyl ether, glycol ether, propylene glycol or dipropylene glycol.

Suitable alcohol ethoxylate can be a linear (preferably C₄₋₁₀) alcohol ethoxylate, a branched alcohol, oxo alcohol ethoxylate. The ethoxylation number may preferably be 1, 2, 3, 4, 5, 6, 7 or 8. In one embodiment, suitable solvent of the present invention comprises at least one alcohol ethoxylate, such as hexyl ethoxylate (e.g., 2-(hexyloxy)ethanol), 2-ethoxyethylalcohol, 2-(2-ethoxyethoxy)ethanol, triethylene glycol monoethyl ether, 2-(hexyloxy)ethanol, 2-(2-hexyloxyethoxy)ethanol, phenoxyethanol or the like.

The solvent can be present at a level of from 0.1 to 40 wt %, from 0.2 to 35 wt %, from 0.5 to 30 wt %, from 1 to 25 wt %, from 2 to 20 wt %, from 3 to 15 wt %, from 4 to 12 wt %, from 5 to 10 wt %, or from 6 to 8 wt %.

In an embodiment, two or three or more solvents are combined, e.g., ethanol and diethylene glycol monobutyl ether, ethanol and diethylene glycol monobutyl ether and propylene glycol, ethanol and propylene glycol, or isopropanol and dipropylene glycol are combined.

In one embodiment, the cleaning composition comprise one or more alcohol ethoxylate. For example, the cleaning composition comprise a hexyl ethoxylate (e.g., 2-(hexyloxy)ethanol), and further comprises one or more other solvents selected from dipropylene glycol monobutyl ether, diethylene glycol ethyl ether, triethylene glycol methyl ether, triethylene glycol ethyl ether, triethylene glycol butyl ether, ethylene glycol monobutyl ether, diethylene glycol monobutyl ether, triethylene glycol monobutyl ether, dipropylene glycol mono butyl ether, diethylene glycol monobutyl ether, 2-ethoxyethylalcohol, 2-(2-ethoxyethoxy)ethanol, triethylene glycol monoethyl ether, 2-(hexyloxy)ethanol, 2-(2-hexyloxyethoxy)ethanol, phenoxyethanol, ethanol, propanol, isopropanol and butanol, isobutanol, ethylene glycol, diethylene glycol, triethylene glycol, propylene glycol and dipropylene glycol.

In one embodiment, the cleaning composition comprise one or more lower alkyl glycol ether solvent. For example, the cleaning composition comprise ethylene glycol monohexyl ether (C6EO1), and further comprises one or more ester solvents selected from dipropylene glycol monobutyl ether, diethylene glycol ethyl ether, triethylene glycol methyl ether, triethylene glycol ethyl ether, triethylene glycol butyl ether, ethylene glycol monobutyl ether, diethylene glycol monobutyl ether, triethylene glycol monobutyl ether, dipropylene glycol mono butyl ether, diethylene glycol monobutyl ether, 2-ethoxyethylalcohol, 2-(2-ethoxyethoxy)ethanol, triethylene glycol monoethyl ether, 2-(hexyloxy)ethanol, 2-(2-hexyloxyethoxy)ethanol and phenoxyethanol.

In one embodiment, the cleaning composition comprises two solvents: ethylene glycol monohexyl ether (C6EO1) and dipropylene glycol monobutyl ether (DPGBE). Preferably, the ratio of the two solvents in weight are in a range of 1:10 to 10:1, e.g., in a range of 1:5 to 5:1, 1:2 to 3:1, e.g., about 1:1.

### Stabilizing Agents

Stabilizing agents that can be used in the cleaning composition include, but are not limited to: primary aliphatic amines, betaines, borate, calcium ions, sodium citrate, citric acid, sodium formate, glycerine, malonic acid, organic diacids, polyols, propylene glycol, and mixtures thereof. Exemplary ranges of the stabilizing agent include may be 0-20 wt %, e.g., between about 0.5 wt % to about 15 wt % and between about 2 wt % to about 10 wt %. In one embodiment, the stabilizing agent is sodium citrate or sodium carbonate.

In an embodiment of the cleaning composition, the amount of stabilizing agent is preferably between about 0.01 wt % and about 5 wt %; more preferably between about 0.05 wt % and about 2.5 wt %; most preferably between about 0.1 wt % and about 1 wt %

### Soap

The compositions of the invention may also contain soap. Without being limited by theory, it may be desirable to include soap as it acts in part as a surfactant and in part as a builder and may be useful for suppression of foam. Any soap known in the art for use in laundry detergents may be utilized. In one embodiment, the compositions contain from 0 wt % to 20 wt %, from 0.5 wt % to 20 wt %, from 4 wt % to 10 wt %, or from 4 wt % to 7 wt % of soap.

Examples of soap useful herein include oleic acid soaps, palmitic acid soaps, palm kernel fatty acid soaps, and mixtures thereof. Typical soaps are in the form of mixtures of fatty acid soaps having different chain lengths and degrees of substitution. One such mixture is topped palm kernel fatty acid.

In a preferred embodiment the composition comprises coco fatty acid. Other suitable fatty acids are saturated and/or unsaturated and can be obtained from natural sources such a plant or animal esters (e.g., palm kernel oil, palm oil, coconut oil, babassu oil, safflower oil, tall oil, castor oil, tallow and fish oils, grease, and mixtures thereof), or synthetically prepared (e.g., via the oxidation of petroleum or by hydrogenation of carbon monoxide via the Fisher Tropsch process).

### Hydrotropes

The compositions of the present invention may also comprise one or more hydrotropes. A hydrotrope is a compound that solubilizes hydrophobic compounds in aqueous solutions (or oppositely, polar substances in a non-polar environment). Typically, hydrotropes have both hydrophilic and a hydrophobic character (so-called amphiphilic properties as known from surfactants); however, the molecular structure of hydrotropes generally do not favor spontaneous self-aggregation, see e.g. review by Hodgdon and Kaler (2007), Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Use of hydrotropes in detergent or cleaning compositions allow for example more concentrated formulations of surfactants (as in the process of compacting liquid detergents by removing water) without inducing undesired phenomena such as phase separation or high viscosity.

The detergent composition of the invention may contain from 0 to 10 wt %, such as from 0 to 5 wt %, 0.5 to 5 wt %, or from 3% to 5 wt %, of a hydrotrope. Any hydrotrope known in the art for use in detergents may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Builders and Co-Builder

The compositions of the present invention may also comprise one or more builders, co-builders, builder systems or a mixture thereof. When a builder is used, the cleaning composition will typically comprise from 0 to 60 wt %, at least 1 wt %, from 2 to 60 wt % or from 5 to 10 wt % builder. In a dish wash cleaning composition, the level of builder is typically e.g. 5 to 45 wt % or 10 to 35 wt %. The composition may be substantially free of builder; substantially free means “no deliberately added” zeolite and/or phosphate. Typical zeolite builders include zeolite A, zeolite P and zeolite MAP. A typical phosphate builder is sodium tri-polyphosphate.

The builder and/or co-builder may particularly be a chelating agent that forms water-soluble complexes with Ca and Mg. Any builder and/or co-builder known in the art for use in detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), iminodiethanol (DEA) and 2,2′,2″-nitrilotriethanol (TEA), and carboxymethylinulin (CMI), and combinations thereof.

The composition may include a co-builder alone, or in combination with a builder, e.g. a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), etheylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diylbis(phosphonic acid) (HEDP), ethylenediaminetetrakis(methylene)tetrakis(phosphonic acid) (EDTMPA), diethylenetriaminepentakis(methylene)pentakis(phosphonic acid) (DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl) aspartic acid (SMAS), N-(2-sulfoethyl) aspartic acid (SEAS), N-(2-sulfomethyl) glutamic acid (SMGL), N-(2-sulfoethyl) glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N, N-diacetic acid (SLDA), taurine-N, N-diacetic acid (TUDA) and sulfomethyl-N,N-diacetic acid (SMDA), N-(hydroxyethyl)-ethylidenediaminetriacetate (HEDTA), diethanolglycine (DEG), Diethylenetriamine Penta (Methylene Phosphonic acid) (DTPMP), aminotris(methylenephosphonic acid) (ATMP), and combinations and salts thereof. Further exemplary builders and/or co-builders are described in, e.g., WO09/102854, U.S. Pat. No. 5,977,053.

### Chelating Agents and Crystal Growth Inhibitors

The compositions of the invention may also contain a chelating agent and/or a crystal growth inhibitor. Suitable molecules include copper, iron and/or manganese chelating agents and mixtures thereof. Suitable molecules include DTPA (Diethylene triamine pentaacetic acid), HEDP (Hydroxyethane diphosphonic acid), DTPMP (Diethylene triamine penta(methylene phosphonic acid)), 1,2-Dihydroxybenzene-3,5-disulfonic acid disodium salt hydrate, ethylenediamine, diethylene triamine, ethylenediaminedisuccinic acid (EDDS), N-hydroxyethylethylenediaminetri-acetic acid (HEDTA), triethylenetetraaminehexaacetic acid (TTHA), N-hydroxyethyliminodiacetic acid (HEIDA), dihydroxyethylglycine (DHEG), ethylenediaminetetrapropionic acid (EDTP), carboxymethyl inulin and 2-Phosphonobutane 1,2,4-tricarboxylic acid (Bayhibit® AM) and derivatives thereof. Typically, the composition may comprise from 0.005 to 15 wt % or from 3.0 to 10 wt % chelating agent or crystal growth inhibitor.

### Bleach Components

The composition of the invention may also comprise a bleach component. The bleach component suitable for incorporation in compositions of the invention or use in methods of the invention comprises one or a mixture of more than one bleach component. Suitable bleach components include bleaching catalysts, photobleaches, bleach activators, hydrogen peroxide, sources of hydrogen peroxide, pre-formed peracids and mixtures thereof. In general, when a bleach component is used, the compositions of the present invention may comprise from 0 to 30 wt %, from 0.00001 to 90 wt %, 0.0001 to 50 wt %, from 0.001 to 25 wt % or from 1 to 20 wt %. Examples of suitable bleach components include:
(1) Pre-formed peracids: Suitable preformed peracids include, but are not limited to, compounds selected from the group consisting of pre-formed peroxyacids or salts thereof, typically either a peroxycarboxylic acid or salt thereof, or a peroxysulphonic acid or salt thereof. Preferably such bleach components may be present in the compositions of the invention in an amount from 0.01 to 50 wt % or from 0.1 to 20 wt %.
(2) Sources of hydrogen peroxide include e.g., inorganic perhydrate salts, including alkali metal salts such as sodium salts of perborate (usually mono- or tetra-hydrate), percarbonate, persulphate, perphosphate, persilicate salts and mixtures thereof. Preferably such bleach components may be present in the compositions of the invention in an amount of 0.01 to 50 wt % or 0.1 to 20 wt %.
(3) The term bleach activator is meant herein as a compound which reacts with hydrogen peroxide to form a peracid via perhydrolysis. The peracid thus formed constitutes the activated bleach. Suitable bleach activators to be used herein include those belonging to the class of esters, amides, imides or anhydrides. Suitable bleach activators are those having R—(C═O)-L wherein R is an alkyl group, optionally branched, having, when the bleach activator is hydrophobic, from 6 to 14 carbon atoms, or from 8 to 12 carbon atoms and, when the bleach activator is hydrophilic, less than 6 carbon atoms or less than 4 carbon atoms; and L is leaving group. Examples of suitable leaving groups are benzoic acid and derivatives thereof—especially benzene sulphonate. Suitable bleach activators include dodecanoyl oxybenzene sulphonate, decanoyl oxybenzene sulphonate, decanoyl oxybenzoic acid or salts thereof, 3,5,5-trimethyl hexanoyloxybenzene sulphonate, tetraacetyl ethylene diamine (TAED), sodium 4-[(3,5,5-trimethylhexanoyl)oxy]benzene-1-sulfonate (ISONOBS), 4-(dodecanoyloxy)benzene-1-sulfonate (LOBS), 4-(decanoyloxy)benzene-1-sulfonate, 4-(decanoyloxy)benzoate (DOBS or DOBA), 4-(nonanoyloxy)benzene-1-sulfonate (NOBS), and/or those disclosed in WO98/17767. A family of bleach activators is disclosed in EP624154 and particularly preferred in that family is acetyl triethyl citrate (ATC). Alternatively, the bleaching system may comprise peroxyacids of, for example, the amide, imide, or sulfone type. The bleaching system may also comprise peracids such as 6-(phthalimido)peroxyhexanoic acid (PAP). Suitable bleach activators are also disclosed in WO98/17767. While any suitable bleach activator may be employed, in one aspect of the invention the subject cleaning composition may comprise NOBS, TAED or mixtures thereof. When present, the peracid and/or bleach activator is generally present in the composition in an amount of 0.1 to 60 wt %, 0.5 to 40 wt % or 0.6 to 10 wt % based on cleaning composition.
(4) Diacyl peroxides (DAP)—preferred diacyl peroxide bleaching species include those selected from diacyl peroxides of the general formula: R¹—C(O)—OO—(O)C—R², in which R¹ represents a C₆-C₁₈ alkyl, preferably C₆-C₁₂ alkyl group containing a linear chain of at least 5 carbon atoms and optionally containing one or more substituents (e.g. —N⁺(CH₃)₃, —COOH or —CN) and/or one or more interrupting moieties (e.g. —CONH— or —CH═CH—) interpolated between adjacent carbon atoms of the alkyl radical, and R² represents an aliphatic group compatible with a peroxide moiety, such that R¹ and R² together contain a total of 8 to 30 carbon atoms. In one preferred aspect R and R2 are linear unsubstituted C₆-C₁₂ alkyl chains. In one embodiment the DAP may be asymmetric, such that preferably the hydrolysis of R1 acyl group is rapid to generate peracid, but the hydrolysis of R2 acyl group is slow.

The tetraacyl peroxide bleaching species is preferably selected from tetraacyl peroxides of the general formula: R³—C(O)—OO—C(O)—(CH₂)n-C(O)—OO—C(O)—R3, in which R³ represents a C₁-C₉ alkyl, or C₃-C₇, group and n represents an integer from 2 to 12, or 4 to 10 inclusive.

Preferably, the diacyl and/or tetraacyl peroxide bleaching species is present in an amount sufficient to provide at least 0.5 ppm, at least 10 ppm, or at least 50 ppm by weight of the wash liquor. In a preferred embodiment, the bleaching species is present in an amount sufficient to provide from 0.5 to 300 ppm, from 30 to 150 ppm by weight of the wash liquor.

Preferably the bleach component comprises a bleach catalyst. When present, the peracid and/or bleach activator is generally present in the composition in an amount of from 0.1 to 60 wt %, from 0.5 to 40 wt % or from 0.6 to 10 wt % based on the composition. One or more hydrophobic peracids or precursors thereof may be used in combination with one or more hydrophilic peracid or precursor thereof.

Exemplary bleaching systems are also described, e.g. in WO2007/087258, WO2007/087244, WO2007/087259 and WO2007/087242.

### Defoaming Agents

The cleaning composition can include a defoaming agent to reduce the stability of foam and reduce foaming. When the composition includes a defoaming agent, the defoaming agent can be provided in an amount of between about 0.01 wt % and about 3 wt %.

Examples of defoaming agents that can be used in the composition includes ethylene oxide/propylene oxide block copolymers such as those available under the name Pluronic N3, silicone compounds such as silica dispersed in polydimethylsiloxane, polydimethylsiloxane, and functionalized polydimethylsiloxane such as those available under the name Abil B9952, fatty amides, hydrocarbon waxes, fatty acids, fatty esters, fatty alcohols, fatty acid soaps, ethoxylates, mineral oils, polyethylene glycol esters, alkyl phosphate esters such as monostearyl phosphate, and the like.

### Lipases

A composition of the invention comprises, besides a surfactant or a surfactant system and a solvent also a lipase. The lipase may be any lipase. In an embodiment, the lipase is of microbial origin. In an embodiment the lipase is of bacterial origin. In a preferred embodiment, the lipase is of fungal origin, such as from a filamentous fungus or a yeast.

Examples of lipases include lipases from Thermomyces, e.g. from T. lanuginosus (previously named Humicola lanuginosa) as described in EP258068 and EP305216, cutinase from Humicola, e.g. H. insolens (WO96/13580), lipase from Absidia reflexa disclosed in US 2009/0221033 A1 (SEQ ID NO: 3), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412, WO13/033318), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

Preferred commercial lipase products include Lipolase™, Lipex™; Lipolex™, Lipoclean™, Lipex Evity 100L, Lipex Evity 105T, Lipex Evity 200L (Novozymes A/S), Lumafast (originally from Genencor), Preferenz L100 (Danisco US Inc.), and Lipomax (originally from Gist-Brocades).

When present in a cleaning composition, the lipase of the invention may be present at levels of at least 0.0001 mg of enzyme protein per gram of the composition, at least 0.001 mg of enzyme protein, at least 0.006 mg of enzyme protein, at least 0.008 mg of enzyme protein, at least 0.01 mg of enzyme protein, at least 0.1 mg of enzyme protein, at least 0.5 mg of enzyme protein, at least 1 mg of enzyme protein, at least 2 mg of enzyme protein, at least 5 mg of enzyme protein, at least 10 mg of enzyme protein, or at least 20 mg of enzyme protein.

Lipase of SEQ ID NO: 1 —Thermomyces lanuginosus Lipase (TLL)

In a preferred embodiment, the enzyme product of the invention or cleaning composition of the invention comprise a lipase derived from a strain of Thermomyces, in particular a strain of Thermomyces lanuginosus (synonym Humicola lanuginosa) or a variant thereof. In a specific embodiment, the lipase is the one shown in SEQ ID NO: 1 or a variant thereof.

In an embodiment, the lipase is:
i) a lipase having at least 60%, at least 65%, at least 70%, at least 75%, at least 80%, at least 85%, at least 90%, at least 95% identity, at least 96%, at least 97%, at least 98%, or at least 99%, or 100% sequence identity to SEQ ID NO: 1;
ii) a variant of a parent lipase having lipase activity having at least 60%, at least 70%, at least 80%, at least 90%, at least 91%, at least 92%, at least 93%, at least 94%, at least 95%, at least 96%, at least 97%, at least 98%, or at least 99%, but less than 100% sequence identity to the lipase shown as SEQ ID NO: 1;
iii) a fragment of the lipase in (i) or (ii) having lipase activity, wherein the variant comprises substitutions at positions corresponding to T231R+N233R and at least one or more (e.g., several) of D96E, D111A, D254S, G163K, P256T, G91T and G38A of SEQ ID NO: 1.

In a specific embodiment, the lipase, used in the composition of the invention, is a variant of a parent lipase, wherein the variant has lipase activity, has at least 60%, in particular at least 65%, at least 70%, at least 75% at least 80%, at least 85%, at least 90%, at least 95%, at least 96%, at least 97%, at least 98%, at least 99% but less than 100% sequence identity with SEQ ID NO: 1, and comprises substitutions selected from the group of:

$$\begin{matrix}D 96 E + T 231 R + N 233 R ; \\ N 33 Q + D 96 E + T 231 R + N 233 R ; \\ N 33 Q + D 111 A + T 231 R + N 233 R ; \\ N 33 Q + T 231 R + N 233 R + P 256 T ; \\ N 33 Q + G 38 A + G 91 T + G 163 K + T 231 R + N 233 R + D 254 S ; \\ N 33 Q + G 38 A + G 91 T + D 96 E + D 111 A + G 163 K + T 231 R + N 233 R + D 254 S + P 256 T ; \\ D 27 R + N 33 Q + G 38 A + D 96 E + D 111 A + G 163 K + T 231 R + N 233 R + D 254 S + P 256 T ; \\ D 27 R + N 33 Q + G 38 A + G 91 T + D 96 E + D 111 A + G 163 K + T 231 R + N 233 R + P 256 T ; \\ D 27 R + N 33 Q + G 38 A + G 91 T + D 96 E + D 111 A + G 163 K + T 231 R + N 233 R + D 254 S ; \\ D 27 R + G 38 A + G 91 T + D 96 E + D 111 A + G 163 K + T 231 R + N 233 R + D 254 S + P 256 T ; \\ D 96 E + T 231 R + N 233 R + D 254 S ; \\ T 231 R + N 233 R + D 254 S + P 256 T ; \\ G 163 K + T 231 R + N 233 R + D 254 S ; \\ D 27 R + N 33 Q + G 38 A + G 91 T + D 96 E + G 163 K + T 231 R + N 233 R + D 254 S + P 256 T ; \\ D 27 R + G 91 T + D 96 E + D 111 A + G 163 K + T 231 R + N 233 R + D 254 S + P 256 T ; \\ D 96 E + G 163 K + T 231 R + N 233 R + D 254 S ; \\ D 27 R + G 163 K + T 231 R + N 233 R + D 254 S ; \\ D 27 R + G 38 A + G 91 T + D 96 E + D 111 A + G 163 K + T 231 R + N 233 R + D 254 S ; \\ D 27 R + G 38 A + G 91 T + D 96 E + G 163 K + T 231 R + N 233 R + D 254 S + P 256 T ; \\ D 27 R + G 38 A + D 96 E + D 111 A + G 163 K + T 231 R + N 233 R + D 254 S + P 256 T ; \\ D 27 R + D 96 E + G 163 K + T 231 R + N 233 R + D 254 S ; \\ D 27 R + D 96 E + D 111 A + G 163 K + T 231 R + N 233 R + D 254 S + P 256 T ; \\ D 27 R + G 38 A + D 96 E + G 163 K + T 231 R + N 233 R + D 254 S + P 256 T \\ D 111 A + G 163 K + T 231 R + N 233 R + D 254 S + P 256 T ; \\ D 111 A + T 231 R + N 233 R ; \\ D 111 A + T 231 R + N 233 R + D 254 S + P 256 T ; \\ D 27 R + D 96 E + D 111 A + G 163 K + T 231 R + N 233 R ; \\ D 27 R + D 96 E + D 111 A + T 231 R + N 233 R ; \\ D 27 R + N 33 Q + G 38 A + D 96 E + D 111 A + T 231 R + N 233 R + D 254 S + P 256 T ; \\ D 27 R + G 38 A + D 96 E + D 111 A + G 163 K + E 210 Q + T 231 R + N 233 R + D 254 S + P 256 T ; \\ D 27 R + T 231 R + N 233 R + D 254 S + P 256 T ; \\ D 96 E + D 111 A + G 163 K + T 231 R + N 233 R ; \\ D 96 E + D 111 A + G 163 K + T 231 R + N 233 R + D 254 S + P 256 T ; \\ D 96 E + D 111 A + G 163 K + T 231 R + N 233 R + P 256 T ; \\ D 96 E + D 111 A + T 231 R + N 233 R ; \\ D 96 E + D 111 A + T 231 R + N 233 R + D 254 S ; \\ D 96 E + D 111 A + T 231 R + N 233 R + D 254 S + P 256 T \\ D 96 E + D 111 A + T 231 R + N 233 R + P 256 T ; \\ D 96 E + G 163 K + T 231 R + N 233 R + D 254 S + P 256 T ; \\ D 96 E + T 231 R + N 233 R + D 254 S + P 256 T ; \\ D 96 E + T 231 R + N 233 R + P 256 T ; \\ G 38 A + D 96 E + D 111 A + T 231 R + N 233 R ; \\ G 38 A + D 96 E + D 111 A + G 163 K + T 231 R + N 233 R + D 254 S + P 256 T ; \\ G 91 T + D 96 E + D 111 A + T 231 R + N 233 R ; \\ G 91 T + D 96 E + T 231 R + N 233 R ; \\ G 91 T + T 231 R + N 233 R + D 254 S + P 256 T ; \\ N 33 Q + D 96 E + D 111 A + G 163 K + T 231 R + N 233 R + D 254 S + P 256 T ; \\ T 231 R + N 233 R + D 254 S + P 256 T ; \\ T 231 R + N 233 R + P 256 T .\end{matrix}$$

In another embodiment, the lipase is a variant of a parent lipase, wherein said variant
(a) comprises a modification in at least one position corresponding to positions E1, V2, N33, F51, E56, L69, K98, V176, H198, E210, Y220, L227, and K237 of SEQ ID NO: 1; and optionally further comprises a modification in at least one position corresponding to positions D27, G38, D96, D111, G163, T231, N233, D254, and P256 of SEQ ID NO: 1;
(b) has a sequence identity of at least 60%, at least 65%, at least 70%, at least 75% at least 80%, at least 85%, at least 90%, at least 95%, at least 96%, at least 97%, at least 98%, at least 99% but less than 100% to SEQ ID NO: 1;
(c) has lipase activity.

In an embodiment, the lipase is a variant of a parent lipase, wherein the parent lipase is selected from the group consisting of:
a) a polypeptide having at least 60%, at least 65%, at least 70%, at least 75%, at least 80%, at least 85%, at least 90%, at least 95% identity, at least 96%, at least 97%, at least 98%, or at least 99%, or 100% sequence identity to SEQ ID NO: 1;
b) a fragment of the polypeptide of SEQ ID NO: 1.

In an embodiment, the lipase is a variant having lipase activity and having at least 60%, at least 65%, at least 70%, at least 75%, at least 80%, at least 85%, at least 90%, at least 95% identity, at least 96%, at least 97%, at least 98%, or at least 99%, but less than 100% sequence identity to SEQ ID NO: 1.

In an embodiment, the lipase variant comprises a modification in at least one of the following positions corresponding to: E1, V2, D27, N33, G38, F51, E56, L69, D96, K98, D111, G163, V176, H198, E210, Y220, L227, T231, N233, K237, D254, and P256, wherein numbering is according to SEQ ID NO: 1. More preferably, the lipase variant comprises at least one of the following modifications corresponding to: E1C, V2Y, D27R, N33K, N33Q, G38A, F51V, E56K, L69R, D96E, D96L, K98I, K98Q, D111A, G163K, V176L, H198S, E210K, Y220F, L227G, T231R, N233R, N233C, K237C, D254S, and P256T, wherein numbering is according to SEQ ID NO: 1.

In an embodiment, the said lipase variant further comprises one of the substitutions selected from the group of: S54T, S83T, G91A, A150G, I255A, and E239C.

In a preferred embodiment, the lipase variant comprises substitutions corresponding to E1C+N233C in SEQ ID NO: 1 and optionally one or more additional substitutions.

In a specific embodiment, the variant has lipase activity, has at least 60%, at least 65%, at least 70%, at least 75% at least 80%, at least 85%, at least 90%, at least 95%, at least 96%, at least 97%, at least 98%, at least 99% but less than 100% sequence identity with SEQ ID NO: 1 and comprises or consists of substitutions corresponding to one of the following set of substitutions using SEQ ID NO: 1 for numbering:

E1C + H198L + N233C
E1C + H198G + N233C
E1C + L69V + N233C
E1C + L69T + N233C
E1C + L69S + N233C
E1C + L69H + N233C
E1C + L69F + N233C
E1C + L69C + N233C
E1C + H198Y + N233C
E1C + H198T + N233C
E1C + H198G + N233C
E1C + L227F + N233C
E1C + L227R + N233C
E1C + E210T + N233C
E1C + E210N + N233C
E1C + V176M + N233C
E1C + K98T + N233C
E1C + K98E + N233C
E1C + E56S + N233C
E1C + E56Q + N233C
E1C + E56R + N233C
E1C + F51M + N233C
E1C + D27R + F51Y + N233C
E1C + V2I + N233C
E1C + V2N + N233C
E1C + V2K + N233C
E1C + V2A + N233C
E1C + D96L + N233C
E1C + L69R + N233C
E1C + V2Y + N233C
E1C + N233C + P256T
E1C + N233C + D254S
E1C + T231R + N233C
E1C + H198S + N233C
E1C + D111A + N233C
E1C + D96E + N233C
E1C + G38A + N233C
E1C + N33Q + N233C
E1C + N33K + N233C
E1C + E210A + N233C
E1C + E210Q + N233C
E1C + E210R + N233C
E1C + H198D + N233C
E1C + K98R + N233C
E1C + K98V + N233C
E1C + F51L + N233C
E1C + F51I + N233C
E1C + K237C
E1C + L227G + N233C
E1C + E210K + N233C
E1C + V176L + N233C
E1C + K98Q + N233C
E1C + E56K + N233C
E1C + L147S + N233C + D254S
E1C + Y220F + N233C
E1C + K98I + N233C
E1C + N233C
E1C + D27R + F51I + E56R + K98E + T231R + N233C
E1C + D27R + F51I + E56R + K98E + T231R + N233C + D254S
E1C + D27R + G38A + F51L + K98I + D111A + G163S + H198S + Y220F + T231R + N233C + P256T
E1C + D27R + G38A + F51L + D96E + K98I + D111A + G163K + H198S + Y220F + T231R + N233C +
D254S + P256T
E1C + D27R + G38R + F51L + D96E + K98I + D111A + G163K + H198S + Y220F + T231R + N233C +
D254S + P256T
E1C + D27R + F51L + D96I + K98I + D111A + G163K + H198S + Y220F + T231R + N233C + P256T
E1C + D27R + F51L + D96E + K98I + D111A + G163S + H198S + Y220F + T231R + N233C + P256T
E1C + D27R + F51L + D96E + K98I + D111A + G163K + H198S + Y220F + T231R + N233C + P256T
E1C + D27R + G38A + F51I + D96E + K98I + D111A + G163K + H198S + Y220F + T231R + N233C +
D254S + P256T
E1C + D27R + G38A + F51V + D96E + K98I + D111A + G163K + H198S + Y220F + T231R + N233C +
P256T
E1C + D27R + F51V + D96E + K98I + D111A + G163K + H198S + Y220F + T231R + N233C + P256T
E1C + D27R + F51V + D96E + K98I + D111A + G163S + H198S + Y220F + T231R + N233C + D254S +
P256T
E1C + D27R + F51V + D96I + K98I + D111A + G163K + H198S + Y220F + T231R + N233C + D254S +
P256T
E1C + D27R + F51V + K98I + D111A + G163K + H198S + Y220F + T231R + N233C + D254S + P256T
E1C + D27R + F51V + D96E + K98I + D111A + G163K + H198S + Y220F + T231R + N233C + D254S +
P256T
E1C + D27R + G38A + F51V + D96E + K98I + D111A + G163K + H198S + Y220F + T231R + N233C +
D254S + P256T
E1C + F51V + D96E + K98I + D111A + G163S + H198S + Y220F + T231R + N233C + P256T
E1C + F51L + D96E + K98I + D111A + G163K + H198S + Y220F + T231R + N233C + D254S + P256T
E1C + G38A + F51V + D96E + K98I + D111A + G163K + H198S + Y220F + T231R + N233C + P256T
E1C + F51V + D96E + K98I + D111A + G163K + H198S + Y220F + T231R + N233C + P256T
E1C + F51V + D96I + K98I + D111A + G163K + H198S + Y220F + T231R + N233C + D254S + P256T
E1C + F51V + K98I + D111A + G163K + H19S + Y220F + T231R + N233C + D254S + P256T
E1C + F51I + D96E + K98I + D111A + G163K + H198S + Y220F + T231R + N233C + D254S + P256T
E1C + D27R + F51L + D96E + K98I + D111A + G163K + H198S + Y220F + T231R + N233C + D254S +
P256T
E1C + D27R + N33K + G38A + F51V + D96E + K98I + D111A + G163K + H198S + Y220F + T231R + N233C
E1C + G38R + F51V + D96E + K98I + D111A + G163K + H198S + Y220F + T231R + N233C + D254S +
P256T
E1C + F51V + D96E + K98I + D111A + G163K + H198S + Y220F + T231R + N233C + D254S + P256T
E1C + G38A + F51V + D96E + K98I + D111A + G163K + H198S + Y220F + T231R + N233C + D254S +
P256T
E1C + D27R + G38R + F51V + D96E + K98I + D111A + G163K + H198S + Y220F + T231R + N233C +
D254S + P256T

### Lipase of SEQ ID NO: 2

In a preferred embodiment, the cleaning composition of the invention comprise the lipase shown in SEQ ID NO: 2 or a variant thereof.

In an embodiment, the lipase is:
i) a lipase having at least 60%, at least 65%, at least 70%, at least 75%, at least 80%, at least 85%, at least 90%, at least 95% identity, at least 96%, at least 97%, at least 98%, or at least 99%, or 100%/sequence identity to SEQ ID NO: 2;
ii) a variant of a parent lipase having lipase activity having at least 60%, at least 70%, at least 80%, at least 90%, at least 91%, at least 92%, at least 93%, at least 940, at least 95%, at least 96%, at least 97%, at least 98%, or at least 99%, but less than 100% sequence identity to the lipase shown as SEQ ID NO: 2;
iii) a fragment of the lipase in (i) or (ii) having lipase activity,
wherein the variant comprises:
(a) a substitution corresponding to:
D1E,Q; A4R; D5Q; L7Y; N8D,E,Q; K11E,Q; R24E,N,Q; N26Q; D27N,Q; A30N; N33Q; T35N; N39D,Q; A46Q; D48Q; F51I,V; L52N, E56N; N57Q; G59S; V60S,M, D62Q; D70Q; N71Q; N73S; I86F,P; N88D,Q; I90R; L93F; N94Q,R,S; D96H,I,Q,S; V98I; N101D,Q,R; S105R; R108E,Q; D111Q; D122Q; D130Q; D158Q; N162D,E; Y164S; D165Q; D167Q; A173Q; N178D; A180E; N200Q; R209Q; Q210E; F211A,N,S,T,Y; G212D,E,R; L227G, V228E,P; R231Q; R232E,Q; R233K,N,Q; T244E; N250D; I255A; A257S; L264A, V, I; and/or
(b) substitutions corresponding to:
A4E+F211V; A4E+L227G; A4E+T252A; D122N+L124S; D165N+D167S; D96N+V98S; E45N+A47S; E87K+F95Y; E87R+N94D; F211V+L227G; F211V+L264A; F211V+T252A; I238C+G246C; L227A+L264A; L227G+I269W; L264A+I269W; N250P+T252I; S105D+R108G; T123N+R125S; T252A+I269W; T252A+L264A; T252A+L264I; T252A+L264P; T252A+L264Q; T252A+L264S; T252A+L264T; T72N+K74S; V60M+L227G; and/or
(c) substitutions corresponding to:
A40N+T252A+L264A; A46N+T252A+L264A; A46N+T252A+L264A; A46R+T252A+L264A; D130H+N250P+T252I; D1A+T252A+L264A; D1C+T252A+L264A; D1F+T252A+L264A; D1G+T252A+L264A; D1H+T252A+L264A; D1L+T252A+L264A; D1M+T252A+L264A; D1G+T252A+L264A; D1R+T252A+L264A; D1W+T252A+L264A; D1Y+T252A+L264A; D5R+T252A+L264A; D62R+T252A+L264A; F10L+T252A+L264A; F10M+T252A+L264A; F211V+L264A+I269W; F211V+T252A+L264A; F51G+T252A+L264A; F51K+T252A+L264A; G106E+N250P+T252I; G65A+T252A+L264A; G65W+T252A+L264A; H198I+T252A+L264A; H198N+P256T+A257I; K74G+T252A+L264A; L12H+N250P+T252I; L227G+T252A+L264A; L75A+T252A+L264A; L75K+T252A+L264A; L75Y+T252A+L264A; L7F+T252A+L264A; N250P+T252I+I255D; N39S+T252A+L264A; N8K+T252A+L264A; N8R+T252A+L264A; N94D+T252A+L264A; Q15M+T252A+L264A; R108Q+R179E+G212E; R232N+T252A+L264A; S37H+N250P+T252I; S3R+T252A+L264A; T50H+T252A+L264A; T50L+T252A+L264A; T50M+T252A+L264A; T50W+T252A+L264A; T50Y+T252A+L264A; V228R+T252A+L264A; V63C+T252A+L264A; V63E+T252A+L264A; V63G+T252A+L264A; V63I+T252A+L264A; V63L+T252A+L264A; V63Q+T252A+L264A; V63S+T252A+L264A; A19T+T252A+L264A; A19S+T252A+L264A; K11L+T252A+L264A; A20V+T252A+L264A; A20T+T252A+L264A; S17C+T252A+L264A; I34S+T252A+L264A; T32P+T252A+L264A; N26A+T252A+L264A; N26W+T252A+L264A; N26K+T252A+L264A; S37V+T252A+L264A; S37Y+T252A+L264A; S37E+T252A+L264A; D27E+T252A+L264A; A38S+T252A+L264A; T72I+T252A+L264A; T72V+T252A+L264A; V60T+T252A+L264A; L43G+T252A+L264A; N33V+T252A+L264A; N33F+T252A+L264A; N33D+T252A+L264A; P42S+T252A+L264A; A47G+T252A+L264A; A47R+T252A+L264A; G31V+T252A+L264A; A46F+T252A+L264A; A46F+T252A+L264A; A46G+T252A+L264A; A40H+T252A+L264A; A46K+T252A+L264A; D62G+T252A+L264A; D62A+T252A+L264A; F66K+T252A+L264A; A49V+T252A+L264A; T50A+T252A+L264A; F51H+T252A+L264A; A49G+T252A+L264A; V63M+T252A+L264A; F51L+T252A+L264A; T50N+T252A+L264A; V63T+T252A+L264A; F51P+T252A+L264A; A49S+T252A+L264A; A49Q+T252A+L264A; V63A+T252A+L264A; S54R+T252A+L264A; F51Y+T252A+L264A; S54D+T252A+L264A; T64S+T252A+L264A; S54C+T252A+L264A; F66N+T252A+L264A; L52W+T252A+L264A; L52T+T252A+L264A; A68V+T252A+L264A; N57S+T252A+L264A; L67Y+T252A+L264A; V69Q+T252A+L264A; S58Y+T252A+L264A; N71C+T252A+L264A; D70R+T252A+L264A; V60M+T252A+L264A; N71G+T252A+L264A; V69E+T252A+L264A; V69K+T252A+L264A; N71D+T252A+L264A; N71T+T252A+L264A; V60A+T252A+L264A; V60W+T252A+L264A; G61A+T252A+L264A; V60G+T252A+L264A; T72G+T252A+L264A; V60L+T252A+L264A; A4R+R233N+T252A; A4R+R233N+L264A; R233N+T252A+L264A; A4R+V60M+L227G; A4R+L227G+R233N; A4R+V60M+R233N; V60M+L227G+R233N; V60M+L227G+L264V; V60M+L227G+L264I; V60M+L227G+T252A; A4R+L227A+L264A; G23A+N250P+T252I; V60K+N250P+T252I; L97V+N250P+T252I; A150G+N250P+T252I; V202L+N250P+T252I; V228P+N250P+T252I; L227G+N250P+T252I; F211G+N250P+T252; V142I+N250P+T252I; V60M+L227G+V228Q; A4L+T252A+L264A; T114E+T252A+L264A; G156A+T252A+L264A; L168E+T252A+L264A; and/or
(d) substitutions corresponding to:
A4K+R231T+T252A+L264A; A4K+R232V+T252A+L264A; L227G+V228A+T252A+L264I; L7F+L227G+T252A+L264A; N250P+T252I+D254N+P256S; Q249N+N250P+N251S+T252I; T244N+G246S+N250P+T252I; T91A+N92D+D96L+V98Q; T91A+V228L+T252A+L264S; V202C+N250P+T252I+P253C; V60M+T91A+T252A+L264A; W221C+G246C+N250P+T252I; D1C+R233C+T252A+L264A; V60M+D99N+N101S+L227G; V60M+S119N+A121S+L227G; V60M+R125N+A127S+L227G; D1G+T252A+P256T+L264A; V60M+L227G+V228R+L264T; N39D+V60M+L227G+P256T; N73G+T252A+L264A; and/or
(e) substitutions corresponding to:
T91A+H198N+D254S+P256T+A257I; T91A+T252A+I255L+P256K+L264A; V60M+L227G+V228L+T252A+L264Y; V60M+L227V+V228P+T252A+L264I; V60M+T91A+L227R+T252A+L264V; N33Q+V60M+G163N+D165S+L227G; R24E+A180E+N250D+T252A+L264A; and/or V60M+T91A+L227V+T252A+L264M; and/or
(f) substitutions corresponding to:
L7F+T91A+A150G+L154V+T252A+L264A; and/or
(g) substitutions corresponding to:
D1C+V202C+R233C+I238C+G245C+T252A+P253C+L264A; and/or
A4R+I90V+N94E+D96L+N101P+R233N+T252A+L264A
   of the polypeptide shown as SEQ ID NO: 2.

In an embodiment the lipase variant further comprises one or more substitutions corresponding to: A4E; A20T; P29S; A46Q; S58N; T91A; N92D; L93F,I; S105D,E,N; R179G,Q; N200R; Y220F; L227G; R231K; T244E; Q249E; T252A,S,V; N25S; A38T; R84S; N94D; V98Q; N101K; D130H; D137G; R232K; T244A,N,K; A249G,R; N250P; T252I; D254S; P256T; A257I; L264A; N94V; F95V; L97S; N101E; S105K; D129G; A134S; V187I; Q188H; R209Q; Q210D; G212S; D234R; G240D; N248D,E; Q249D,G; L264V; T267A; I269F; A28V; V60E,I,M; V63I; E87Q; N92D; V98Q; S105G; R179K; V228L; N248K; Q249D; I255G; L264P,Y; A4Q; L7F; A46K; Y53F; V60K; E87K; Y138F; A157V; Y194F; H198I; Y213F; L227V, V228A; and/or I255L in SEQ ID NO: 2 or using SEQ ID NO: 2 for numbering.

In an embodiment, the lipase variant comprises two substitutions corresponding to:
A20T+L93F; A4E+A46Q; F51I+T244E; L227G+R233N; L227G+T244E; L227G+T252A; L93I+V98I; N101D+S105D; Q210E+Q249E; R179Q+G212E; R231K+R233K; R233N+T252A; S105D+G212E; S105E+R108Q; S105N+G212D; S105N+G212E; S58N+V60S; T244E+T252A; T252A+I255A; T252S+I255A; and/or T91A+V98I in SEQ ID NO: 2 or using SEQ ID NO: 2 for numbering.

In an embodiment, the lipase comprises substitutions corresponding to:
A4R+R233N; K223Q+R232Q; Q210E+N250D; R108Q+G212E; R24Q+N250D; and/or R24Q+Q210E; in SEQ ID NO: 2 or using SEQ ID NO: 2 for numbering.

In an embodiment, the lipase variant comprises substitutions corresponding to:
A38T+D96H+D137G; A4R+T252A+L264A; D1G+T252A+L264A; D62N+T252A+L264A; D165Q+N250P+T252I; H198S+Y220F+L264A; N101K+S105N+R108E; N94Q+N250P+T252I; Q210E+T244E+Q249G; Q210E+T252A+L264A; R231K+R232K+R233K; S83T+H198S+D254S; R233N+T252A+L264A; A46Q+T252A+L264A; N39D+T252A+L264A; a+L227G; or F51I+T252A+L264A in SEQ ID NO: 2 or using SEQ ID NO: 2 for numbering.

In an embodiment, the lipase variant comprises substitutions corresponding to:
A4R+R233N+T252A+L264A; A4R+V60M+L227G+R231T; A4R+V60M+L227G+R232V; A4R+V60M+L227G+R233N; E87Q+T91A+D96I+V98Q; G109R+Q210E+T244N+Q249E; L227G+R233N+T252A+L264A; L7F+Q210E+T252A+L264A; Q188H+Q210E+T252A+L264A; Q210E+L227G+T252A+L264A; Q210N+G212S+N250P+T252I; V60S+L227G+T252A+L264A; R24E+N33Q+V60M+L227G; or R24E+V228P+T252A+L264A in SEQ ID NO: 2 or using SEQ ID NO: 2 for numbering.

In an embodiment, the lipase variant comprises substitutions corresponding to:
L227G+R233N+T244E+T252A+L264A; S105N+R108Q+D129G+D137G+G212D; L7F+R24E+N39D+T252A+L264A; and/or R24E+V128A+V228E+T252A+L264A; or A20T+G163N+D165S+T252A+L264A in SEQ ID NO: 2 or using SEQ ID NO: 2 for numbering.

In an embodiment, the lipase variant comprises substitutions corresponding to:
L7F+N8K+Q210E+L227G+T252A+L264A; N8D+101K+S105G+R108Q+R179E+G212E; N8D+R209Q+Q210E+T244N+N248K+Q249E; V60E+S83T+T91A+H198S+T252A+L264P; or V60M+T91A+Q210E+V228L+T252A+L264Y in SEQ ID NO: 2 or using SEQ ID NO: 2 for numbering.

In an embodiment, the lipase variant comprises substitutions corresponding to:

$$V 60 K + S 83 T + T 91 A + H 198 I + V 228 L + T 252 A + L 2 \; 64 P ;$$

or V60M+A157V+Q210E+L227V+V228A+T252A+L264V in SEQ ID NO: 2 or using SEQ ID NO: 2 for numbering.

In an embodiment, the lipase variant comprises substitutions corresponding to:

$$D 1 E + A 4 Q + L 7 F + K 11 N + S 37 T + A 46 K + A 133 R + V 142 F + T 170 S + V 202 I + Q 210 E + L 227 G .$$

In a specific preferred embodiment, the lipase has one of the following set of substitutions corresponding to (using SEQ ID NO: 2 for numbering):

A180E
A257S
A46Q
D122Q
D130Q
D158Q
D165Q
D167Q
D1E
D1Q
D27Q
D48Q
D5Q
D70Q
F211A
F211L
F211N
F211S
F211T
I86F
K11Q
L52N
N101Q
N162D
N178D
N250D
N26Q
N33Q
N39D
N39Q
N57Q
N71Q
N8D
N8E
N8Q
N94Q
N94S
Q210E
R108E
R108Q
R209Q
R233K
R24E
R24Q
T244E
V98I
Y164S
A4E + A46Q
A4E + F211V
A4E + L227G
A4E + T252A
A4R + R233N
F211V + L227G
F211V + L264A
F211V + T252A
F51I + T244E
I238C + G246C
L227A + L264A
L227G + I269W
L227G + R233N
L227G + T244E
L227G + T252A
L264A + I269W
Q210E + N250D
R233N + T252A
S105D + R108G
S105E + R108Q
S105N + G212D
S58N + V60S
T244E + T252A
T252A + I269W
T252A + L264A
T252A + L264I
T252A + L264P
T252A + L264Q
T252A + L264S
T252A + L264T
V60M + L227G
A38T + D96H + D137G
A40N + T252A + L264A
A46N + T252A + L264A
A46R + T252A + L264A
D1A + T252A + L264A
D1C + T252A + L264A
D1G + T252A + L264A
D1L + T252A + L264A
D1Q + T252A + L264A
D1Y + T252A + L264A
D5R + T252A + L264A
F10L + T252A + L264A
F10M + T252A + L264A
F211V + L264A + I269W
F211V + T252A + L264A
F211V + T252A + L264A
F211V + T252A + L264A
F51G + T252A + L264A
G65A + T252A + L264A
H198I + T252A + L264A
K74G + T252A + L264A
L75A + T252A + L264A
L75K + T252A + L264A
L75Y + T252A + L264A
L7F + T252A + L264A
L7F + T252A + L264A
N101K + S105N + R108E
N39S + T252A + L264A
N8K + T252A + L264A
N8R + T252A + L264A
N94D + T252A + L264A
Q15M + T252A + L264A
Q210E + T244E + Q249G
Q210E + T252A + L264A
Q210E + T252A + L264A
R233N + T252A + L264A
T50H + T252A + L264A
T50L + T252A + L264A
T50M + T252A + L264A
T50W + T252A + L264A
T50Y + T252A + L264A
V63C + T252A + L264A
V63E + T252A + L264A
V63L + T252A + L264A
V63S + T252A + L264A
A4K + R231T + T252A + L264A
A4K + R232V + T252A + L264A
A4R + R233N + T252A + L264A
A4R + V60M + L227G + R231T
A4R + V60M + L227G + R232V
A4R + V60M + L227G + R233N
L227G + R233N + T252A + L264A
Q188H + Q210E + T252A + L264A
Q210E + L227G + T252A + L264A
T91A + V228L + T252A + L264S
V202C + N250P + T252I + P253C
V60M + T91A + T252A + L264A
V60S + L227G + T252A + L264A
L227G + R233N + T244E + T252A + L264A
T91A + T252A + I255L + P256K + L264A
V60M + L227G + V228L + T252A + L264Y
L7F + T91A + A150G + L154V + T252A + L264A
V60K + S83T + T91A + H198I + V228L + T252A + L264P
A19S + T252A + L264A
A20V + T252A + L264A
A20T + T252A + L264A
S17C + T252A + L264A
I34S + T252A + L264A
N26A + T252A + L264A
N26K + T252A + L264A
S37V + T252A + L264A
S37Y + T252A + L264A
S37E + T252A + L264A
D27E + T252A + L264A
A38S + T252A + L264A
T72I + T252A + L264A
T72V + T252A + L264A
V60T + T252A + L264A
L43G + T252A + L264A
N33V + T252A + L264A
N33F + T252A + L264A
N33D + T252A + L264A
P42S + T252A + L264A
A47G + T252A + L264A
A47R + T252A + L264A
A46F + T252A + L264A
A46G + T252A + L264A
A46Q + T252A + L264A
A40H + T252A + L264A
A46K + T252A + L264A
N39D + T252A + L264A
D62G + T252A + L264A
V69Q + T252A + L264A
S58Y + T252A + L264A
N71C + T252A + L264A
D70R + T252A + L264A
N71G + T252A + L264A
V69E + T252A + L264A
V69K + T252A + L264A
N71D + T252A + L264A
N71T + T252A + L264A
V60M + D99N + N101S + L227G
V60M + S119N + A121S + L227G
V60M + R125N + A127S + L227G
L7F + R24E + N39D + T252A + L264A
A20T + G163N + D165S + T252A + L264A
D1G + T252A + P256T + L264A
V228P + T252A + L264A
V60M + L227G + V228R + L264T
T114E + T252A + L264A
G156A + T252A + L264A
L168E + T252A + L264A

### Other Lipases

Other lipases contemplated according to the present invention are those disclosed in the following applications: WO2019/038164, WO2019/121585, WO2019/138121, WO2019/155789, WO2019/155790, WO2019/185519, WO2019/185610, WO2019/185612, WO2019/201636, WO2019/206994, WO2019/215078, WO2019/219903, WO2019/243312 as well as the Absidia sp lipase and the variant thereof disclosed in WO 2021/001400 (all hereby incorporated by reference).

Suitable lipase may include a lipase derived from Geotrichum candidum (also called GCL I). Exemplary GCL I can be those selected from SEQ ID NO: 3, SEQ ID NO: 4, SEQ ID NO: 5, SEQ ID NO: 6 or SEQ ID NO: 7.

In an embodiment, the GCL I comprises the amino acid sequence of SEQ ID NO: 3 or comprises an amino acid sequence having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the polypeptide of SEQ ID NO: 1. In one aspect, the polypeptides differ by up to 10 amino acids, e.g., 1, 2, 3, 4, 5, 6, 7, 8, 9, or 10, from the polypeptide comprising SEQ ID NO: 3.

In an embodiment, the GCL I of SEQ ID NO: 3 comprises a substitution, deletion, and/or insertion at one or more (e.g., several) positions. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

### Additional Enzymes

Besides a lipase enzyme, a composition of the invention may comprise one or more additional enzymes which provide cleaning performance. Examples of suitable enzymes include, but are not limited to, proteases, cellulases, amylases, pectinases, mannanases, pectate lyases, phosphodiesterases (PDEs), deoxyribonucleases (DNases), hexosaminidases, hemicelluloses, or mixtures thereof. A typical combination is an enzyme cocktail that may comprise e.g. a protease and lipase in conjunction with an alpha-amylase, phospholipases, cutinases, pectinases, mannanases, pectate lyases, phosphodiesterases (PDEs), deoxyribonucleases (DNases), xanthanase, dispersin, or mixtures thereof.

When present in a cleaning composition, the aforementioned additional enzymes may be present at levels of at least 0.0001 mg of enzyme protein per gram of the composition, at least 0.001 mg of enzyme protein, at least 0.006 mg of enzyme protein, at least 0.008 mg of enzyme protein, at least 0.01 mg of enzyme protein, at least 0.1 mg of enzyme protein, at least 0.5 mg of enzyme protein, at least 1 mg of enzyme protein, at least 2 mg of enzyme protein, at least 5 mg of enzyme protein, at least 10 mg of enzyme protein, or at least 20 mg of enzyme protein.

In general, the properties of the selected enzyme(s) should be compatible with the selected detergent or cleaning composition, (i.e., pH-optimum, compatibility with other enzymatic and non-enzymatic ingredients, etc.), and the enzyme(s) should be present in effective amounts.

Proteases: In one aspect, suitable proteases may be of any origin, but are preferably of bacterial or fungal origin, optionally in the form of protein engineered or chemically modified mutants. The protease may be an alkaline protease, such as a serine protease or a metalloprotease. A serine protease may for example be of the Si family, such as trypsin, or the S8 family such as a subtilisin. A metalloprotease may for example be a thermolysin, e.g. from the M4 family, or another metalloprotease such as those from the M5, M7 or M8 families.

The term “subtilases” refers to a sub-group of serine proteases according to Siezen et al., Protein Eng. 4 (1991) 719-737 and Siezen et al., Protein Sci. 6 (1997) 501-523. Serine proteases are a subgroup of proteases characterized by having a serine in the active site, which forms a covalent adduct with the substrate. The subtilases may be divided into six subdivisions, the Subtilisin family, the Thermitase family, the Proteinase K family, the Lantibiotic peptidase family, the Kexin family and the Pyrolysin family.

Although proteases suitable for detergent use may be obtained from a variety of organisms, including fungi such as Aspergillus, detergent proteases have generally been obtained from bacteria and in particular from Bacillus. Examples of Bacillus species from which subtilases have been derived include Bacillus lentus, Bacillus alkalophilus, Bacillus subtilis. Bacillus amyloliquefaciens, Bacillus licheniformis, Bacillus pumilus and Bacillus gibsonii. Particular subtilisins include subtilisin lentus, subtilisin Novo, subtilisin Carlsberg, subtilisin BPN', subtilisin 309, subtilisin 147 and subtilisin 168 and e.g. protease PD138 (described in WO 93/18140). Other useful proteases are e.g. those described in WO 01/16285 and WO 02/16547.

Examples of trypsin-like proteases include the Fusarium protease described in WO 94/25583 and WO 2005/040372, and the chymotrypsin proteases derived from Cellumonas described in WO 2005/052161 and WO 2005/052146.

Examples of metalloproteases include the neutral metalloproteases described in WO 2007/044993 such as those derived from Bacillus amyloliquefaciens, as well as e.g. the metalloproteases described in WO 2015/158723 and WO 2016/075078.

Examples of useful proteases are the protease variants described in WO 89/06279 WO 92/19729, WO 96/34946, WO 98/20115, WO 98/20116, WO 99/11768, WO 01/44452, WO 03/006602, WO 2004/003186, WO 2004/041979, WO 2007/006305, WO 2011/036263, WO 2014/207227, WO 2016/087617 and WO 2016/174234. Preferred protease variants may, for example, comprise one or more of the mutations selected from the group consisting of: S3T, V4I, S9R, S9E, A15T, S24G, S24R, K27R, N42R, S55P, G59E, G59D, N60D, N60E, V66A, N74D, S85R, A96S, S97G, S97D, S97A, S97SD, S99E, S99D, S99G, S99M, S99N, S99R, S99H, S101A, V102I, V102Y, V102N, S104A, G116V, G116R, H118D, H118N, A120S, S126L, P127Q, S128A, S154D, A156E, G157D, G157P, S158E, Y161A, R164S, Q176E, N179E, S182E, Q185N, A188P, G189E, V193M, N198D, V1991, Q200L, Y203W, S206G, L211Q, L211D, N212D, N212S, M216S, A226V, K229L, Q230H, Q239R, N246K, S253D, N255W, N255D, N255E, L256E, L256D T268A and R269H, wherein position numbers correspond to positions of the Bacillus lentus protease shown in SEQ ID NO: 1 of WO 2016/001449. Protease variants having one or more of these mutations are preferably variants of the Bacillus lentus protease (Savinase®, also known as subtilisin 309) shown in SEQ ID NO: 1 of WO 2016/001449 or of the Bacillus amyloliquefaciens protease (BPN′) shown in SEQ ID NO: 2 of WO 2016/001449. Such protease variants preferably have at least 80% sequence identity to SEQ ID NO: 1 or to SEQ ID NO: 2 of WO 2016/001449.

Another protease of interest is the alkaline protease from Bacillus lentus DSM 5483, as described for example in WO 91/02792, and variants thereof which are described for example in WO 92/21760, WO 95/23221, EP 1921147, EP 1921148 and WO 2016/096711.

The protease may alternatively be a variant of the TY145 protease having SEQ ID NO: 1 of WO 2004/067737, for example a variant comprising a substitution at one or more positions corresponding to positions 27, 109, 111, 171, 173, 174, 175, 180, 182, 184, 198, 199 and 297 of SEQ ID NO: 1 of WO 2004/067737, wherein said protease variant has a sequence identity of at least 75% but less than 100% to SEQ ID NO: 1 of WO 2004/067737. TY145 variants of interest are described in e.g. WO 2015/014790, WO 2015/014803, WO 2015/014804, WO 2016/097350, WO 2016/097352, WO 2016/097357 and WO 2016/097354.

Suitable commercially available protease enzymes may include those sold under the trade names Alcalase®, Duralase™, Durazym™, Relase®, Relase® Ultra, Savinase®, Savinase® Ultra, Primase™ Polarzyme®, Kannase®, Liquanase®, Liquanase® Ultra, Ovozyme®, Coronase®, Coronase® Ultra, Blaze®, Blaze® Pro, Blaze Evity® 100T, Blaze Evity® 125T, Blaze Evity® 150T, Blaze Evity® 200T, Neutrase®, Everlase®, Esperase®, Progress® Uno, Progress® In and Progress® Excel (Novozymes A/S), those sold under the tradename Maxatase™, Maxacal™, Maxapem®, Purafect® Ox, Purafect® OxP, Puramax®, FN2™, FN3™, FN4^{ex}™, Excellase®, Excellenz™ P1000, Excellenz™ P1250, Eraser™ Preferenz® P100, Preferenz® P300, Purafect Prime, Preferenz P110™, Effectenz P1000™ Purafect®, Effectenz P1050™, Purafect® Ox, Effectenz™ P2000, Purafast™, Properase®, Opticlean™ and Optimase® (Danisco/DuPont), BLAP (sequence shown in FIG. 29 of U.S. Pat. No. 5,352,604) and variants hereof (Henkel AG), and KAP (Bacillus alkalophilus subtilisin) from Kao.

Amylases: In one aspect, suitable amylases may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

$$\begin{matrix}M 197 T ; \\ H 156 Y + A 181 T + N 190 F + A 209 V + Q 264 S ; or \\ G 48 A + T 49 I + G 107 A + H 156 Y + A 181 T + N 190 F + I 201 F + A 209 V + Q 264 S .\end{matrix}$$

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, I206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

$$\begin{matrix}N 128 C + K 178 L + T 182 G + Y 305 R + G 4 \; 75 K ; \\ N 128 C + K178 L + T 182 G + F 202 Y + Y 305 R + D 319 T + G 4 \; 75 K ; \\ S 125 A + N 128 C + K 178 L + T 182 G + Y 305 R + G 4 \; 75 K ; or \\ S 125 A + N 128 C + T 131 I + T 165 I + K 178 L + T 182 G + Y 305 R + G 475 K\end{matrix}$$

wherein the variants are C-terminally truncated and optionally further comprises a substitution at position 243 and/or a deletion at position 180 and/or position 181.

Further suitable amylases are amylases having SEQ ID NO: 1 of WO13184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, I203, S241, R458, T459, D460, G476 and G477. More preferred variants of SEQ ID NO: 1 are those having the substitution in one of more of the following positions: K176L, E187P, N192FYH, M199L, 1203YF, S241QADN, R458N, T459S, D460T, G476K and G477K and/or deletion in position R178 and/or S179 or of T180 and/or G181. Most preferred amylase variants of SEQ ID NO: 1 are those having the substitutions:

$$\begin{matrix}E 187 P + I 203 Y + G 476 K \\ E 187 P + I 203 Y + R 458 N + T 459 S + D 460 T + G 476 K\end{matrix}$$

wherein the variants optionally further comprise a substitution at position 241 and/or a deletion at position 178 and/or position 179.

Further suitable amylases are amylases having SEQ ID NO: 1 of WO10104675 or variants having 90% sequence identity to SEQ ID NO: 1 thereof. Preferred variants of SEQ ID NO: 1 are those having a substitution, a deletion or an insertion in one of more of the following positions: N21, D97, V128 K177, R179, S180, I181, G182, M200, L204, E242, G477 and G478. More preferred variants of SEQ ID NO: 1 are those having the substitution in one of more of the following positions: N21D, D97N, V128I K177L, M200L, L204YF, E242QA, G477K and G478K and/or deletion in position R179 and/or S180 or of I181 and/or G182. Most preferred amylase variants of SEQ ID NO: 1 are those having the substitutions:

$$N 21 D + D 97 N + V 128 I$$

wherein the variants optionally further comprise a substitution at position 200 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Duramyl™, Termamyl™, Fungamyl™, Stainzyme™ Stainzyme Plus™, Natalase™, Liquozyme X and BAN™ (from Novozymes A/S), and Rapidase™ Purastar™/Effectenz™, Powerase, Preferenz S1000, Preferenz S100 and Preferenz S110 (from Genencor International Inc./DuPont).

Cellulases: In one aspect, suitable cellulases include mono-component and mixtures of enzymes of bacterial or fungal origin. Chemically modified or protein engineered mutants are also contemplated. The cellulase may for example be a mono-component or a mixture of mono-component endo-1,4-beta-glucanase also referred to as endoglucanase.

Suitable cellulases include those from the genera Bacillus, Pseudomonas, Humicola, Myceliophthora, Fusarium, Thielavia, Trichoderma, and Acremonium. Exemplary cellulases include a fungal cellulase from Humicola insolens (U.S. Pat. No. 4,435,307) or from Trichoderma, e.g. T. reesei or T. viride. Other suitable cellulases are from Thielavia e.g. Thielavia terrestris as described in WO 96/29397 or the fungal cellulases produced from Myceliophthora thermophila and Fusarium oxysporum disclosed in U.S. Pat. Nos. 5,648,263, 5,691,178, 5,776,757, WO 89/09259 and WO 91/17244. Also relevant are cellulases from Bacillus as described in WO 02/099091 and JP 2000210081. Suitable cellulases are alkaline or neutral cellulases having care benefits. Examples of cellulases are described in EP 0 495 257, EP 0 531 372, WO 96/11262, WO 96/29397, WO 98/08940. Other examples are cellulase variants such as those described in WO 94/07998, EP 0 531 315, U.S. Pat. Nos. 5,457,046, 5,686,593, 5,763,254, WO 95/24471, WO 98/12307.

Other cellulases are endo-beta-1,4-glucanase enzyme having a sequence of at least 97% identity to the amino acid sequence of position 1 to position 773 of SEQ ID NO:2 of WO 2002/099091 or a family 44 xyloglucanase, which a xyloglucanase enzyme having a sequence of at least 60% identity to positions 40-559 of SEQ ID NO: 2 of WO 2001/062903.

Commercially available cellulases include Carezyme®, Carezyme® Premium, Celluzyme®, Celluclean®, Celluclast®, Endolase®, Renozyme®; Whitezyme® Celluclean® Classic, Cellusoft® (Novozymes A/S), Puradax®, Puradax HA, and Puradax EG (available from Genencor International Inc.) and KAC-500(B)™ (Kao Corporation).

Mannanases: In one aspect, suitable mannanases include those of bacterial or fungal origin. Chemically or genetically modified mutants are included. The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 1999/064619. A commercially available mannanase is Mannaway (Novozymes A/S) and Purabrite® (Danisco/DuPont).

Pectate lyases, DNases and/or PDEs: Other preferred enzymes that additionally may be comprised in the composition of the invention include pectate lyases, e.g., one sold under the tradenames Pectawash®, Pectaway®, or Xpect®. Finally, the composition may also comprise a deoxyribonuclease (DNase) and/or a phosphodiesterase (PDE).

In one embodiment, in addition to the lipase of the invention, the cleaning composition further comprises an amylase, e.g. BAN™ (from Novozymes A/S).

In one embodiment, in addition to the lipase of the invention, the cleaning composition further comprises a protease, a dispersin and/or a DNase.

In one embodiment, in addition to the lipase of the invention, the cleaning composition further comprises a protease and a cellulase.

In one embodiment, in addition to the lipase of the invention, the cleaning composition further comprises a protease, an amylase and a cellulase.

### Polymers

The cleaning composition may contain 0.005-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties. Exemplary polymers include (carboxymethyl)cellulose (CMC), poly(vinyl alcohol) (PVA), poly(ethylDeneglycol) or poly(ethylene oxide) (PEG or PEO), ethoxylated poly(ethyleneimine), (carboxymethyl)inulin (CMI), carboxylate polymers and d, and lauryl methacrylate/acrylic acid copolymers, hydrophobically modified CMC (HM-CMC), silicones, copolycarboxylates such as polyacrylates, maleic/acrylic acid copolymers, acrylate/styrene copolymers, poly(aspartic) acipolymers of terephthalic acid and oligomeric glycols, copolymers of poly(ethylene terephthalate) and poly(oxyethene terephthalate) (PET-POET), poly(vinylpyrrolidone) (PVP), poly(vinylimidazole) (PVI), poly(vinylpyridine-N-oxide) (PVPO or PVPNO) and copoly(vinylimidazole/vinylpyrrolidone) (PVPVI). Suitable examples include PVP-K15, PVP-K30, ChromaBond S-400, ChromaBond S-403E and Chromabond S-100 from Ashland Aqualon, and Sokalan® HP 165, Sokalan® HP 50 (Dispersing agent), Sokalan® HP 53 (Dispersing agent), Sokalan® HP 59 (Dispersing agent), Sokalan® HP 56 (dye transfer inhibitor), Sokalan® HP 66 K (dye transfer inhibitor) from BASF. Further exemplary polymers include sulfonated polycarboxylates, polyethylene oxide and polypropylene oxide (PEO-PPO) and diquaternium ethoxy sulfate. Particularly preferred polymer is ethoxylated homopolymer Sokalan® HP 20 from BASF, which helps to prevent redeposition of soil in the wash liquor. Further exemplary polymers include sulfonated polycarboxylates, ethylene oxide-propylene oxide copolymers (PEO-PPO), copolymers of PEG with and vinyl acetate, and diquaternium ethoxy sulfate or quaternized sulfated ethoxylated hexamethylenediamine. Other exemplary polymers are disclosed in, e.g., WO 2006/130575. Salts of the above-mentioned polymers are also contemplated.

### Adjunct Materials

Any detergent adjunct components known in the art may also be utilized. Exemplary adjunct materials may include anti-corrosion agents, anti-shrink agents, anti-soil redeposition agents, anti-wrinkling agents, bactericides, binders, corrosion inhibitors, disintegrates/disintegration agents, dyes, enzyme stabilizers (including boric acid, borates, CMC, and/or polyols such as propylene glycol), foam boosters, foam (suds) regulators, perfumes, soil-suspending agents, softeners, suds suppressors, tarnish inhibitors, and wicking agents, either alone or in combination. The choice of such ingredients is well within the skill of the artisan. The cleaning compositions of the present invention may also include a corrosion inhibitor (or “rust inhibitor”) to prevent rust or corrosion of the hard surface after washing or cleaning.

### Soil Release Polymers

The cleaning compositions of the present invention may also include one or more soil release polymers which aid the removal of soils from a surface to be cleaned. The soil release polymers may for example be nonionic or anionic terephthalate based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore random graft co-polymers are suitable soil release polymers. Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

### Probiotics

The compositions may comprise probiotics (e.g., probiotic spores) such as those described in WO09/043709. Commercially available probiotics include Microvia® and Deep Clean Multi 5×(available from Novozymes A/S).

### Formulation of Cleaning Composition Products

The hard surface cleaning composition of the invention may be formulated in any convenient form, e.g., a liquid, a gel, a paste, a bar, a powder, a tablet, a pouch having one or more compartments, a single or a multi-compartment unit dose form, or a spray form.

Pouches can be configured as single or multicompartments. They can be of any form, shape and material which is suitable for hold the composition, e.g. without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid cleaning composition or part components and/or a liquid cleaning composition or part components separated by the water soluble film.

Detergent ingredients or cleaning components can be separated physically from each other by compartments in water dissolvable pouches or in different layers of tablets. Thereby negative storage interaction between components can be avoided.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent. A liquid or gel detergent may be non-aqueous.

The composition of the present invention may be formulated as a hand dish wash detergent, a professional cleaning detergent, a cleaning-in-place (CIP) detergent or a hard surface cleanser.

When formulated as liquid or spray type, or a paste form, the cleaning composition of the invention is preferred to have a pH of below 11, e.g. in the range of 6-9.5, in the range of 6.5-9, in the range of 6.8-8.5, in the range of 7-8, more preferably have a neutral pH of 6.5-7.5, such as pH 7.0, 7.1 or 7.3.

When formulated as a solid type e.g., a tablet, the cleaning composition of the invention is preferably dissolved by water to form wash liquor before applied to the hard surface to be cleaned. Such wash liquor is preferred to have a pH of below 11, e.g. in the range of 6-9.5, in the range of 6.5-9, in the range of 6.8-8.5, in the range of 7-8, and more preferably in the range of pH 6.5-7.5, such as pH 7.0, 7.1 or 7.3.

### Formulation of Enzyme in Co-Granule

The enzyme of the invention may be formulated as a granule for example as a co-granule that combines one or more enzymes. Each enzyme will then be present in more granules securing a more uniform distribution of enzymes in the detergent. This also reduces the physical segregation of different enzymes due to different particle sizes. Methods for producing multi-enzyme co-granulates for the detergent industry are disclosed in the IP.com disclosure IPCOM000200739D.

Another example of formulation of enzymes by the use of co-granulates is disclosed in WO 2013/188331, which relates to a cleaning composition comprising (a) a multi-enzyme co-granule; (b) less than 10 wt zeolite (anhydrous basis); and (c) less than 10 wt phosphate salt (anhydrous basis), wherein said enzyme co-granule comprises from 10 to 98 wt % moisture sink component and the composition additionally comprises from 20 to 80 wt % detergent moisture sink component. WO 2013/188331 also relates to a method of treating and/or cleaning a surface, comprising the steps of (i) contacting said surface with the cleaning composition as claimed and described herein in an aqueous wash liquor, (ii) rinsing and/or drying the surface.

The multi-enzyme co-granule may comprise an enzyme of the invention and (a) one or more enzymes selected from the group consisting of proteases, cellulases, amylases, pectinases, mannanases, pectate lyases, phosphodiesterases (PDEs), deoxyribonucleases (DNases), hexosaminidases, hemicelluloses, and mixtures thereof.

In some embodiments, the hard surface cleaning composition may comprise components shown in below table 1.

TABLE 1
Components of the exemplary cleaning compositions of the present invention

| Exemplary cleaning composition 1 | | Exemplary cleaning composition 2 | |
|---|---|---|---|
| components | wt % | components | wt % |
| Polyoxyethylene ether of butyl alcohol - PPG26 | 2.84 | MPG | 9.33 |
| Hexyl ethoxylate (C6EOn) | 2.84 | | |
| Phenoxy_ethanol | 0.08 | APG | 1.5 |
| Na_Adipate | 0.1 | AEOS | 1.15 |
| Na₂SO₄ | 0.02 | AEO | 0.9 |
| MEA | 0.01 | Na₂SO₄ | 0.04 |
| GLDA (glutamic acid diacetic acid) | 3 | CitrateNa | 2.6 |
| DPGBE (dipropylene glycol monobutyl ether) | 2.78 | Sodium dialkyl | 1-2 |
| | | sulfosuccinate | |
| AO | 1 | Lipase | 0.01-5 |
| AEOS | 4 | Water | adjust to 100 |
| AEO | 3.2 | | |
| Lipase | 0.01-5 | | |
| Water | adjust | | |
| | to 100 | | |

The present invention is further summarized in the following paragraphs:
1. A hard surface cleaning composition comprising:
(a) 0.1 to 60 wt % of a surfactant system;
(b) 0.1 to 40 wt % of a solvent, wherein the solvent is selected from the group consisting of a lower alkanol, a benzyl alcohol, a lower alkyl ether, a glycol, an aryl glycol ether, a lower alkyl glycol ether, a glycerol ketal, an ester, a hydrocarbon/ester blend, a dibasic ester, alcohol ethoxylate such as a linear alcohol ethoxylate, a branched alcohol or oxo alcohol ethoxylate, and combinations thereof; and optionally
(c) 0.001 to 10 wt % of a lipase.
2. The composition of paragraph 1, wherein the surfactant system comprises at least one surfactant selected from the group consisting of nonionic surfactants, anionic surfactants, cationic surfactants, ampholytic surfactants, zwitterionic surfactants, semi-polar nonionic surfactants, and mixtures thereof.
3. The composition of paragraph 1 or 2, wherein the surfactant system comprises one or more anionic surfactant and/or one or more nonionic surfactant.
4. The composition of any one of paragraphs 1-3, wherein the surfactant system comprises one or more anionic surfactants, preferably linear alkylbenzenesulfonic acid (LAS), alcohol ethersulfate (AEOS) and/or alkyl sulfate (AS), in particular sodium lauryl sulfate (SLS).
5. The composition of any one of paragraphs 1-4, wherein the surfactant system comprises one or more nonionic surfactants, preferably alcohol ethoxylate (AEO), in particular linear alcohol (C₁₂₋₁₅) ethoxylate.
6. The composition of any one of paragraphs 1-5, wherein the anionic surfactant is present at a level of from 0 to 20 wt %, from 0.5 to 15 wt %, 2 to 15 wt %, from 3 to 12 wt %, from 4 to 10 wt %, or from 1 to 5 wt %.
7. The composition of any one of paragraphs 1-6, wherein the composition comprises the anionic surfactants linear alkylbenzenesulfonic acid (LAS) and a nonionic surfactant alcohol ethoxylate (AEO).
8. The composition of any of proceeding paragraphs, wherein the nonionic surfactant is present at a level of from 0.2 to 30 wt %, from 0.5 to 25 wt %, from 1 to 20 wt %, from 2 to 15 wt %, from 3 to 12 wt %, from 4 to 10 wt %, or from 6 to 8 wt %.
9. The composition of any of proceeding paragraphs, wherein the solvent is selected from ethanol, propanol, isopropanol and butanol, isobutanol, ethylene glycol, diethylene glycol, triethylene glycol, propylene glycol, dipropylene glycol, mixed ethylene-propylene glycol ethers, ethylene glycol phenyl ether, propylene glycol phenyl ether, propylene glycol methyl ether, propylene glycol propyl ether, dipropylene glycol methyl ether, tripropylene glycol butyl ether, tripropylene glycol methyl ether, ethylene glycol butyl ether, diethylene glycol methyl ether, diethylene glycol butyl ether, ethylene glycol dimethyl ether, ethylene glycol propyl ether, diethylene glycol ethyl ether, triethylene glycol methyl ether, triethylene glycol ethyl ether, triethylene glycol butyl ether, ethylene glycol monobutyl ether, diethylene glycol monobutyl ether, triethylene glycol monobutyl ether, dipropylene glycol mono butyl ether, diethylene glycol monobutyl ether, hexyl ethoxylate (e.g., ethylene glycol monohexyl ether), 2-ethoxyethylalcohol, 2-(2-ethoxyethoxy)ethanol, triethylene glycol monoethyl ether, 2-(hexyloxy)ethanol, 2-(2-hexyloxyethoxy)ethanol, phenoxyethanol, and combination thereof, e.g. a combination of ethanol and diethylene glycol monobutyl ether, glycol ether, propylene glycol or dipropylene glycol.
10. The composition of any of proceeding paragraphs, wherein the solvent is present at a level of from 0.2 to 35 wt %, from 0.5 to 30 wt %, from 1 to 25 wt %, from 2 to 20 wt %, from 3 to 15 wt %, from 4 to 12 wt %, from 5 to 10 wt %, or from 6 to 8 wt %.
11. The composition of any of proceeding paragraphs, wherein the composition further comprises one or more components selected from the group of builders, chelating agents, dispersants, enzyme stabilizers, catalytic materials, bleach activators, hydrogen peroxide, sources of hydrogen peroxide, preformed peracids, polymeric dispersing agents, suds suppressors, dyes, perfumes, structure elasticizing agents, carriers, hydrotropes, processing aids, and/or pigments.
12. The composition of any of proceeding paragraphs, wherein the composition further comprises an additional enzyme selected from the group consisting of proteases, cellulases, amylases, pectinases, mannanases, pectate lyases, phosphodiesterases (PDEs), deoxyribonucleases (DNases), hexosaminidases, hemicelluloses, and combinations thereof.
13. The composition of any one of paragraphs 1-12, wherein the lipase is of microbial origin, in particular of fungal or bacterial origin.
14. The composition of any one of paragraphs 1-13, wherein the composition comprises a lipase, e.g., a fungal lipase derived from a strain of Thermomyces lanuginosus lipase.
15. The composition of any of paragraphs 1-14, wherein the lipase is
   i) a lipase having at least 60%, at least 65%, at least 70%, at least 75%, at least 80%, at least 85%, at least 90%, at least 95% identity, at least 96%, at least 97%, at least 98%, or at least 99%, or 100% sequence identity to SEQ ID NO: 1, 2, 3, 4. 5, 6 or 7;
   ii) a variant having lipase activity having at least 60%, at least 70%, at least 80%, at least 90%, at least 91%, at least 92%, at least 93%, at least 94%, at least 95%, at least 96%, at least 97%, at least 98%, or at least 99%, but less than 100% sequence identity to the lipase shown as SEQ ID NO: 1, 2, 3, 4. 5, 6 or 7;
   iii) a fragment of the lipase in (i) or (ii) having lipase activity.
16. The composition of any one of paragraph 1-15, wherein the lipase is a variant comprises substitutions at positions corresponding to T231R+N233R and optionally at least one or more of D96E, D111A, D254S, G163K, P256T, G91T and G38A of SEQ ID NO: 1.
17. The composition of any one of paragraphs 1-16, wherein the lipase is a variant of a parent lipase, wherein the variant has lipase activity and has at least 60%, at least 65%, at least 70%, at least 75%, at least 80%, at least 85%, at least 90%, at least 95%, at least 96%, at least 97%, at least 98%, at least 99%, but less than 100% sequence identity with SEQ ID NO: 1, and comprises substitutions at positions corresponding to T231R+N233R and at least one or more of D96E, D111A, D254S, G163K, P256T, G91T and G38A of SEQ ID NO: 1 selected from the group of:

$$\begin{matrix}a . & D 96 E + T 231 R + N 233 R ; \\ b . & N 33 Q + D 96 E + T 231 R + N 233 R ; \\ c . & N 33 Q + D 111 A + T 231 R + N 233 R ; \\ d . & N 33 Q + T 231 R + N 233 R + P 256 T ; \\ e . & N 33 Q + G 38 A + G 91 T + G 163 K + T 231 R + N 233 R + D 254 S ; \\ f . & N 33 Q + G 38 A + G 91 T + D 96 E + D 111 A + G 163 K + T 231 R + N 233 R + D 254 S + P 256 T ; \\ g . & D 27 R + N 33 Q + G 38 A + D 96 E + D 111 A + G 163 K + T 231 R + N 233 R + D 254 S + P 256 T ; \\ h . & D 27 R + N 33 Q + G 38 A + G 91 T + D 96 E + D 111 A + G 163 K + T 231 R + N 233 R + P 256 T ; \\ i . & D 27 R + N 33 Q + G 38 A + G 91 T + D 96 E + D 111 A + G 163 K + T 231 R + N 233 R + D 254 S ; \\ j . & D 27 R + G 38 A + G 91 T + D 96 E + D 111 A + G 163 K + T 231 R + N 233 R + D 254 S + P 256 T ; \\ k . & D 96 E + T 231 R + N 233 R + D 254 S ; \\ l . & T 231 R + N 233 R + D 254 S + P 256 T ; \\ m . & G 163 K + T 231 R + N 233 R + D 254 S ; \\ n . & D 27 R + N 33 Q + G 38 A + G 91 T + D 96 E + G 163 K + T 231 R + N 233 R + D 254 S + P 256 T ; \\ o . & D 27 R + G 91 T + D 96 E + D 111 A + G 163 K + T 231 R + N 233 R + D 254 S + P 256 T ; \\ p . & D 96 E + G 163 K + T 231 R + N 233 R + D 254 S ; \\ q . & D 27 R + G 163 K + T 231 R + N 233 R + D 254 S ; \\ r . & D 27 R + G 38 A + G 91 T + D 96 E + D 111 A + G 163 K + T 231 R + N 233 R + D 254 S ; \\ s . & D 27 R + G 38 A + G 91 T + D 96 E + G 163 K + T 231 R + N 233 R + D 254 S + P 256 T ; \\ t . & D 27 R + G 38 A + D 96 E + D 111 A + G 163 K + T 231 R + N 233 R + D 254 S + P 256 T ; \\ u . & D 27 R + D 96 E + G 163 K + T 231 R + N 233 R + D 254 S ; \\ v . & D 27 R + D 96 E + D 111 A + G 163 K + T 231 R + N 233 R + D 254 S + P 256 T ; \\ w . & D 27 R + G 38 A + D 96 E + G 163 K + T 231 R + N 233 R + D 254 S + P 256 T \\ x . & D 111 A + G 163 K + T 231 R + N 233 R + D 254 S + P 256 T ; \\ y . & D 111 A + T 231 R + N 233 R ; \\ z . & D 111 A + T 231 R + N 233 R + D 254 S + P 256 T ; \\ aa . & D 27 R + D 96 E + D 111 A + G 163 K + T 231 R + N 233 R ; \\ bb . & D 27 R + D 96 E + D 111 A + T 231 R + N 233 R ; \\ cc . & D 27 R + N 33 Q + G 38 A + D 96 E + D 111 A + T 231 R + N 233 R + D 254 S + P 256 T ; \\ dd . & D 27 R + G 38 A + D 96 E + D 111 A + G 163 K + E 210 Q + T 231 R + N 233 R + D 254 S + P 256 T ; \\ ee . & D 27 R + T 231 R + N 233 R + D 254 S + P 256 T ; \\ ff . & D 96 E + D 111 A + G 163 K + T 231 R + N 233 R ; \\ gg . & D 96 E + D 111 A + G 163 K + T 231 R + N 233 R + D 254 S + P 256 T ; \\ hh . & D 96 E + D 111 A + G 163 K + T 231 R + N 233 R + P 256 T ; \\ ii . & D 96 E + D 111 A + T 231 R + N 233 R ; \\ jj . & D 96 E + D 111 A + T 231 R + N 233 R + D 254 S ; \\ kk . & D 96 E + D 111 A + T 231 R + N 233 R + D 254 S + P 256 T \\ ll . & D 96 E + D 111 A + T 231 R + N 233 R + P 256 T ; \\ m m . & D 96 E + G 163 K + T 231 R + N 233 R + D 254 S + P 256 T ; \\ nn . & D 96 E + T 231 R + N 233 R + D 254 S + P 256 T ; \\ oo . & D 96 E + T 231 R + N 233 R + P 256 T ; \\ pp . & G 38 A + D 96 E + D 111 A + T 231 R + N 233 R ; \\ qq . & G 38 A + D 96 E + D 111 A + G 163 K + T 231 R + N 233 R + D 254 S + P 256 T ; \\ rr . & G 91 T + D 96 E + D 111 A + T 231 R + N 233 R ; \\ ss . & G 91 T + D 96 E + T 231 R + N 233 R ; \\ tt . & G 91 T + T 231 R + N 233 R + D 254 S + P 256 T ; \\ uu . & N 33 Q + D 96 E + D 111 A + G 163 K + T 231 R + N 233 R + D 254 S + P 256 T ; \\ vv . & T 231 R + N 233 R + D 254 S + P 256 T ; \\ ww . & T 231 R + N 233 R + P 256 T .\end{matrix}$$

18. The composition of any one of paragraphs 1-17, wherein the lipase is a variant of a parent lipase, wherein said variant
   (a) comprises a modification in at least one position corresponding to positions E1, V2, N33, F51, E56, L69, K98, V176, H198, E210, Y220, L227, and K237 of SEQ ID NO: 1; and optionally further comprises a modification in at least one position corresponding to positions D27, G38, D96, D111, G163, T231, N233, D254, and P256 of SEQ ID NO: 1;
   (b) has a sequence identity of at least 60% but less than 100% to SEQ ID NO: 1;
   (c) has lipase activity.
19. The composition of any one of paragraphs 1-18, wherein said lipase variant comprises a modification in at least one of the following positions: E1, V2, D27, N33, G38, F51, E56, L69, D96, K98, D111, G163, V176, H198, E210, Y220, L227, T231, N233, K237, D254, and P256, wherein numbering is according to SEQ ID NO: 1.
20. The composition of any one of paragraph 18 or 19, wherein said lipase variant comprises at least one of the following modifications: E1C, V2Y, D27R, N33K, N33Q, G38A, F51V, E56K, L69R, D96E, D96L, K98I, K98Q, D111A, G163K, V176L, H198S, E210K, Y220F, L227G, T231R, N233R, N233C, K237C, D254S, and P256T, wherein numbering is according to SEQ ID NO: 1.
21. The composition of any one of paragraphs 18-20, wherein said lipase variant further comprises one of the substitutions selected from the group of: S54T, S83T, G91A, A150G, I255A, and E239C, wherein numbering is according to SEQ ID NO: 1.
22. The composition of any one of paragraphs 18-21, wherein the lipase variant comprises substitutions E1C+N233C and one or more additional substitutions, wherein numbering is according to SEQ ID NO: 1.
23. The composition of any one of paragraphs 18-22, wherein the variant has lipase activity and has at least 60%, at least 65%, at least 70%, at least 75%, at least 80%, at least 85%, at least 90%, at least 95%, at least 96%, at least 97%, at least 98%, at least 99%, but less than 100% sequence identity with SEQ ID NO: 1 and comprises or consists of substitutions corresponding to one of the following set of substitutions using SEQ ID NO: 1 for numbering:

$$\begin{matrix}E 1 C + H 198 L + N 233 C \\ E 1 C + H 198 G + N 233 C \\ E 1 C + L 69 V + N 233 C \\ E 1 C + L 69 T + N 233 C \\ E 1 C + L 69 S + N 233 C \\ E 1 C + L 69 H + N 233 C \\ E 1 C + L 69 F + N 233 C \\ E 1 C + L 69 C + N 233 C \\ E 1 C + H 198 Y + N 233 C \\ E 1 C + H 198 T + N 233 C \\ E 1 C + H 198 G + N 233 C \\ E 1 C + L 227 F + N 233 C \\ E 1 C + L 227 R + N 233 C \\ E 1 C + E 210 T + N 233 C \\ E 1 C + E 210 N + N 233 C \\ E 1 C + V 176 M + N 233 C \\ E 1 C + K 98 T + N 233 C \\ E 1 C + K 98 E + N 233 C \\ E 1 C + E 56 S + N 233 C \\ E 1 C + E 56 Q + N 233 C \\ E 1 C + E 56 R + N 233 C \\ E 1 C + F 51 M + N 233 C \\ E 1 C + D 27 R + F 51 Y + N 233 C \\ E 1 C + V 2 I + N 233 C \\ E 1 C + V 2 N + N 233 C \\ E 1 C + V 2 K + N 233 C \\ E 1 C + V 2 A + N 233 C \\ E 1 C + D 96 L + N 233 C \\ E 1 C + L 69 R + N 233 C \\ E 1 C + V 2 Y + N 233 C \\ E 1 C + N 233 C + P 256 T \\ E 1 C + N 233 C + D 254 S \\ E 1 C + T 231 R + N 233 C \\ E 1 C + H 198 S + N 233 C \\ E 1 C + D 111 A + N 233 C \\ E 1 C + D 96 E + N 233 C \\ E 1 C + G 38 A + N 233 C \\ E 1 C + N 33 Q + N 233 C \\ E 1 C + N 33 K + N 233 C \\ E 1 C + E 210 A + N 233 C \\ E 1 C + E 210 Q + N 233 C \\ E 1 C + E 210 R + N 233 C \\ E 1 C + H 198 D + N 233 C \\ E 1 C + K 98 R + N 233 C \\ E 1 C + K 98 V + N 233 C \\ E 1 C + F 51 L + N 233 C \\ E 1 C + F 51 I + N 233 C \\ E 1 C + K 237 C \\ E 1 C + L 227 G + N 233 C \\ E 1 C + E 210 K + N 233 C \\ E 1 C + V 176 L + N 233 C \\ E 1 C + K 98 Q + N 233 C \\ E 1 C + E 56 K + N 233 C \\ E 1 C + L 147 S + N 233 C + D 254 S \\ E 1 C + Y 220 F + N 233 C \\ E 1 C + K 98 I + N 233 C \\ E 1 C + N 233 C \\ E 1 C + D 27 R + F 51 I + E 56 R + K 98 E + T 231 R + N 233 C \\ E 1 C + D 27 R + F 51 I + E 56 R + K 98 E + T 231 R + N 233 C + D 254 S \\ E 1 C + D 27 R + G 38 A + F 51 L + K 98 I + D 111 A + G 163 S + H 198 S + Y 220 F + T 231 R + N 233 C + P 256 T \\ E 1 C + D 27 R + G 38 A + F 51 L + D 96 E + K 98 I + D 111 A + G 163 K + H 198 S + Y 220 F + T 231 R + N 233 C + D 254 S + P 256 T \\ E 1 C + D 27 R + G 38 R + F 51 L + D 96 E + K 98 I + D 111 A + G 163 K + H 198 S + Y 220 F + T 231 R + N 233 C + D 254 S + P 256 T \\ E 1 C + D 27 R + F 51 L + D 96 I + K 98 I + D 111 A + G 163 K + H 198 S + Y 220 F + T 231 R + N 233 C + P 256 T \\ E 1 C + D 27 R + F 51 L + D 96 E + K 98 I + D 111 A + G 163 S + H 198 S + Y 220 F + T 231 R + N 233 C + P 256 T \\ E 1 C + D 27 R + F 51 L + D 96 E + K 98 I + D 111 A + G 163 K + H 198 S + Y 220 F + T 231 R + N 233 C + P 256 T \\ E 1 C + D 27 R + G 38 A + F 51 I + D 96 E + K 98 I + D 111 A + G 163 K + H 198 S + Y 220 F + T 231 R + N 233 C + D 254 S + P 256 T \\ E 1 C + D 27 R + G 38 A + F 51 V + D 96 E + K 98 I + D 111 A + G 163 K + H 198 S + Y 220 F + T 231 R + N 233 C + P 256 T \\ E 1 C + D 27 R + F 51 V + D 96 E + K 98 I + D 111 A + G 163 K + H 198 S + Y 220 F + T 231 R + N 233 C + P 256 T \\ E 1 C + D 27 R + F 51 V + D 96 E + K 98 I + D 111 A + G 163 S + H 198 S + Y 220 F + T 231 R + N 233 C + D 254 S + P 256 T \\ E 1 C + D 27 R + F 51 V + D 96 I + K 98 I + D 111 A + G 163 K + H 198 S + Y 220 F + T 231 R + N 233 C + D 254 S + P 256 T \\ E 1 C + D 27 R + F 51 V + K 98 I + D 111 A + G 163 K + H 198 S + Y 220 F + T 231 R + N 233 C + D 254 S + P 256 T \\ E 1 C + D 27 R + F 51 V + D 96 E + K 98 I + D 111 A + G 163 K + H 198 S + Y 220 F + T 231 R + N 233 C + D 254 S + P 256 T \\ E 1 C + D 27 R + G 38 A + F 51 V + D 96 E + K 98 I + D 111 A + G 163 K + H 198 S + Y 220 F + T 231 R + N 233 C + D 254 S + P 256 T \\ E 1 C + F 51 V + D 96 E + K 98 I + D 111 A + G 163 S + H 198 S + Y 220 F + T 231 R + N 233 C + P 256 T \\ E 1 C + F 51 L + D 96 E + K 98 I + D 111 A + G 163 K + H 198 S + Y 220 F + T 231 R + N 233 C + D 254 S + P 256 T \\ E 1 C + G 38 A + F 51 V + D 96 E + K 98 I + D 111 A + G 163 K + H 198 S + Y 220 F + T 231 R + N 233 C + P 256 T \\ E 1 C + F 51 V + D 96 E + K 98 I + D 111 A + G 163 K + H 198 S + Y 220 F + T 231 R + N 233 C + P 256 T \\ E 1 C + F 51 V + D 96 I + K 98 I + D 111 A + G 163 K + H 198 S + Y 220 F + T 231 R + N 233 C + D 254 S + P 256 T \\ E 1 C + F 51 V + K 98 I + D 111 A + G 163 K + H 19 S + Y 220 F + T 231 R + N 233 C + D 254 S + P 256 T \\ E 1 C + F 51 I + D 96 E + K 98 I + D 111 A + G 163 K + H 198 S + Y 220 F + T 231 R + N 233 C + D 254 S + P 256 T \\ E 1 C + D 27 R + F 51 L + D 96 E + K 98 I + D 111 A + G 163 K + H 198 S + Y 220 F + T 231 R + N 233 C + D 254 S + P 256 T \\ E 1 C + D 27 R + N 33 K + G 38 A + F 51 V + D 96 E + K 98 I + D 111 A + G 163 K + H 198 S + Y 220 F + T 231 R + N 233 C \\ E 1 C + G 38 R + F 51 V + D 96 E + K 98 I + D 111 A + G 163 K + H 198 S + Y 220 F + T 231 R + N 233 C + D 254 S + P 256 T \\ E 1 C + F 51 V + D 96 E + K 98 I + D 111 A + G 163 K + H 198 S + Y 220 F + T 231 R + N 233 C + D 254 S + P 256 T \\ E 1 C + G 38 A + F 51 V + D 96 E + K 98 I + D 111 A + G 163 K + H 198 S + Y 220 F + T 231 R + N 233 C + D 254 S + P 256 T \\ E 1 C + D 27 R + G 38 R + F 51 V + D 96 E + K 98 I + D 111 A + G 163 K + H 198 S + Y 220 F + T 231 R + N 233 C + D 254 S + P 256 T .\end{matrix}$$

24. The composition of paragraphs 1-23, wherein the lipase is
   i) a lipase having at least 60%, at least 65%, at least 70%, at least 75%, at least 80%, at least 85%, at least 90%, at least 95% identity, at least 96%, at least 97%, at least 98%, or at least 99%, or 100% sequence identity to SEQ ID NO: 2;
   ii) a variant having lipase activity having at least 60%, at least 70%, at least 80%, at least 90%, at least 91%, at least 92%, at least 93%, at least 94%, at least 95%, at least 96%, at least 97%, at least 98%, or at least 99%, but less than 100% sequence identity to the lipase shown as SEQ ID NO: 2;
   iii) a fragment of the lipase in (i) or (ii) having lipase activity.
25. The composition of paragraph 24, wherein the composition comprises or consists of the amino acid of SEQ ID NO: 2.
26. The composition of paragraph 24 or 25, wherein the lipase is a variant of SEQ ID NO: 2, wherein the variant comprises:
   (a) a substitution corresponding to: D1E,Q; A4R; D5Q; L7Y; N8D,E,Q; K11E,Q; R24E,N,Q; N26Q; D27N,Q; A30N; N33Q; T35N; N39D,Q; A46Q; D48Q; F51I,V; L52N, E56N; N57Q; G59S; V60S,M, D62Q; D70Q; N71Q; N73S; I86F,P; N88D,Q; I90R; L93F; N94Q,R,S; D96H,I,Q,S; V98I; N101D,Q,R; S105R; R108E,Q; D111Q; D122Q; D130Q; D158Q; N162D,E; Y164S; D165Q; D167Q; A173Q; N178D; A180E; N200Q; R209Q; Q210E; F211A,N,S,T,Y; G212D,E,R; L227G, V228E,P; R231Q; R232E,Q; R233K,N,Q; T244E; N250D; I255A; A257S; L264A, V, I; and/or
   (b) substitutions corresponding to: A4E+F211V; A4E+L227G; A4E+T252A; D122N+L124S; D165N+D167S; D96N+V98S; E45N+A47S; E87K+F95Y; E87R+N94D; F211V+L227G; F211V+L264A; F211V+T252A; I238C+G246C; L227A+L264A; L227G+I269W; L264A+I269W; N250P+T252I; S105D+R108G; T123N+R125S; T252A+I269W; T252A+L264A; T252A+L264I; T252A+L264P; T252A+L264Q; T252A+L264S; T252A+L264T; T72N+K74S; V60M+L227G; and/or
   (c) substitutions corresponding to:
   A40N+T252A+L264A; A46N+T252A+L264A; A46N+T252A+L264A; A46R+T252A+L264A; D130H+N250P+T252I; D1A+T252A+L264A; D1C+T252A+L264A; D1F+T252A+L264A; D1G+T252A+L264A; D1H+T252A+L264A; D1L+T252A+L264A; D1M+T252A+L264A; D1G+T252A+L264A; D1R+T252A+L264A; D1W+T252A+L264A; D1Y+T252A+L264A; D5R+T252A+L264A; D62R+T252A+L264A; F10L+T252A+L264A; F10M+T252A+L264A; F211V+L264A+I269W; F211V+T252A+L264A; F51G+T252A+L264A; F51K+T252A+L264A; G106E+N250P+T252I; G65A+T252A+L264A; G65W+T252A+L264A; H198I+T252A+L264A; H198N+P256T+A257I; K74G+T252A+L264A; L12H+N250P+T252I; L227G+T252A+L264A; L75A+T252A+L264A; L75K+T252A+L264A; L75Y+T252A+L264A; L7F+T252A+L264A; N250P+T252I+I255D; N39S+T252A+L264A; N8K+T252A+L264A; N8R+T252A+L264A; N94D+T252A+L264A; Q15M+T252A+L264A; R108Q+R179E+G212E; R232N+T252A+L264A; S37H+N250P+T252I; S3R+T252A+L264A; T50H+T252A+L264A; T50L+T252A+L264A; T50M+T252A+L264A; T50W+T252A+L264A; T50Y+T252A+L264A; V228R+T252A+L264A; V63C+T252A+L264A; V63E+T252A+L264A; V63G+T252A+L264A; V63I+T252A+L264A; V63L+T252A+L264A; V63Q+T252A+L264A; V63S+T252A+L264A; A19T+T252A+L264A; A19S+T252A+L264A; K11L+T252A+L264A; A20V+T252A+L264A; A20T+T252A+L264A; S17C+T252A+L264A; I34S+T252A+L264A; T32P+T252A+L264A; N26A+T252A+L264A; N26W+T252A+L264A; N26K+T252A+L264A; S37V+T252A+L264A; S37Y+T252A+L264A; S37E+T252A+L264A; D27E+T252A+L264A; A38S+T252A+L264A; T72I+T252A+L264A; T72V+T252A+L264A; V60T+T252A+L264A; L43G+T252A+L264A; N33V+T252A+L264A; N33F+T252A+L264A; N33D+T252A+L264A; P42S+T252A+L264A; A47G+T252A+L264A; A47R+T252A+L264A; G31V+T252A+L264A; A46F+T252A+L264A; A46F+T252A+L264A; A46G+T252A+L264A; A40H+T252A+L264A; A46K+T252A+L264A; D62G+T252A+L264A; D62A+T252A+L264A; F66K+T252A+L264A; A49V+T252A+L264A; T50A+T252A+L264A; F51H+T252A+L264A; A49G+T252A+L264A; V63M+T252A+L264A; F51L+T252A+L264A; T50N+T252A+L264A; V63T+T252A+L264A; F51P+T252A+L264A; A49S+T252A+L264A; A49Q+T252A+L264A; V63A+T252A+L264A; S54R+T252A+L264A; F51Y+T252A+L264A; S54D+T252A+L264A; T64S+T252A+L264A; S54C+T252A+L264A; F66N+T252A+L264A; L52W+T252A+L264A; L52T+T252A+L264A; A68V+T252A+L264A; N57S+T252A+L264A; L67Y+T252A+L264A; V69Q+T252A+L264A; S58Y+T252A+L264A; N71C+T252A+L264A; D70R+T252A+L264A; V60M+T252A+L264A; N71G+T252A+L264A; V69E+T252A+L264A; V69K+T252A+L264A; N71D+T252A+L264A; N71T+T252A+L264A; V60A+T252A+L264A; V60W+T252A+L264A; G61A+T252A+L264A; V60G+T252A+L264A; T72G+T252A+L264A; V60L+T252A+L264A; A4R+R233N+T252A; A4R+R233N+L264A; R233N+T252A+L264A; A4R+V60M+L227G; A4R+L227G+R233N; A4R+V60M+R233N; V60M+L227G+R233N; V60M+L227G+L264V; V60M+L227G+L264I; V60M+L227G+T252A; A4R+L227A+L264A; G23A+N250P+T252I; V60K+N250P+T252I; L97V+N250P+T252I; A150G+N250P+T252I; V202L+N250P+T252I; V228P+N250P+T252I; L227G+N250P+T252I; F211G+N250P+T252I; V142I+N250P+T252I; V60M+L227G+V228Q; A4L+T252A+L264A; T114E+T252A+L264A; G156A+T252A+L264A; L168E+T252A+L264A; N73G+T252A+L264A; and/or
   (d) substitutions corresponding to:
   A4K+R231T+T252A+L264A; A4K+R232V+T252A+L264A; L227G+V228A+T252A+L264I; L7F+L227G+T252A+L264A; N250P+T252I+D254N+P256S; Q249N+N250P+N251S+T252I; T244N+G246S+N250P+T252I; T91A+N92D+D96L+V98Q; T91A+V228L+T252A+L264S; V202C+N250P+T252I+P253C; V60M+T91A+T252A+L264A; W221C+G246C+N250P+T252I; D1C+R233C+T252A+L264A; V60M+D99N+N101S+L227G; V60M+S119N+A121S+L227G; V60M+R125N+A127S+L227G; D1G+T252A+P256T+L264A; V60M+L227G+V228R+L264T; and/or
   (e) substitutions corresponding to:
   T91A+H198N+D254S+P256T+A257I; T91A+T252A+I255L+P256K+L264A; V60M+L227G+V228L+T252A+L264Y; V60M+L227V+V228P+T252A+L264I; V60M+T91A+L227R+T252A+L264V; N33Q+V60M+G163N+D165S+L227G; R24E+A180E+N250D+T252A+L264A; and/or V60M+T91A+L227V+T252A+L264M; and/or
   (f) substitutions corresponding to:
   L7F+T91A+A150G+L154V+T252A+L264A; and/or (g) substitutions corresponding to: D1C+V202C+R233C+I238C+G245C+T252A+P253C+L264A; and/or A4R+I90V+N94E+D96L+N101P+R233N+T252A+L264A; of the polypeptide shown as SEQ ID NO: 2.
27. The composition of paragraphs 26, further comprising one or more substitutions corresponding to: A4E; A20T; P29S; A46Q; S58N; T91A; N92D; L93F,I; S105D,E,N; R179G,Q; N200R; Y220F; L227G; R231K; T244E; Q249E; T252A,S,V; N25S; A38T; R84S; N94D; V98Q; N101K; D130H; D137G; R232K; T244A,N,K; A249G,R; N250P; T252I; D254S; P256T; A257I; L264A; N94V; F95V; L97S; N101E; S105K; D129G; A134S; V187I; Q188H; R209Q; Q210D; G212S; D234R; G240D; N248D,E; Q249D,G; L264V; T267A; I269F; A28V; V60E,I,M; V63I; E87Q; N92D; V98Q; S105G; R179K; V228L; N248K; Q249D; I255G; L264P,Y; A4Q; L7F; A46K; Y53F; V60K; E87K; Y138F; A157V;Y194F; H198I; Y213F; L227V, V228A; and/or I255L (using SEQ ID NO: 2 for numbering).
28. The composition of any of paragraphs 26 or 27, comprising substitutions corresponding to: A20T+L93F; A4E+A46Q; F51I+T244E; L227G+R233N; L227G+T244E; L227G+T252A; L93I+V98I; N101D+S105D; Q210E+Q249E; R179Q+G212E; R231K+R233K; R233N+T252A; S105D+G212E; S105E+R108Q; S105N+G212D; S105N+G212E; S58N+V60S; T244E+T252A; T252A+I255A; T252S+I255A; and/or T91A+V98I (using SEQ ID NO: 2 for numbering). 29. The composition of any of paragraphs 26-28, comprising substitutions corresponding to: A4R+R233N; K223Q+R232Q; Q210E+N250D; R108Q+G212E; R24Q+N250D and/or R24Q+Q210E (using SEQ ID NO: 2 for numbering).
30. The composition of any of paragraphs 26-29, comprising substitutions corresponding to: A38T+D96H+D137G; A4R+T252A+L264A; D1G+T252A+L264A; D62N+T252A+L264A; D165Q+N250P+T252I; H198S+Y220F+L264A; N101K+S105N+R108E; N94Q+N250P+T252I; Q210E+T244E+Q249G; Q210E+T252A+L264A; R231K+R232K+R233K; S83T+H198S+D254S; R233N+T252A+L264A; A46Q+T252A+L264A; N39D+T252A+L264A; R24E+V60M+L227G; and/or F51I+T252A+L264A (using SEQ ID NO: 2 for numbering).
31. The composition of any of paragraphs 26-30, comprising substitutions corresponding to: A4R+R233N+T252A+L264A; A4R+V60M+L227G+R231T; A4R+V60M+L227G+R232V; A4R+V60M+L227G+R233N; E87Q+T91A+D96I+V98Q; G109R+Q210E+T244N+Q249E; L227G+R233N+T252A+L264A; L7F+Q210E+T252A+L264A; Q188H+Q210E+T252A+L264A; Q210E+L227G+T252A+L264A; Q210N+G212S+N250P+T252I; V60S+L227G+T252A+L264A; R24E+N33Q+V60M+L227G; and/or R24E+V228P+T252A+L264A (using SEQ ID NO: 2 for numbering).
32. The composition of any of paragraphs 26-31, comprising substitutions corresponding to: L227G+R233N+T244E+T252A+L264A; S105N+R108Q+D129G+D137G+G212D; L7F+R24E+N39D+T252A+L264A; R24E+V128A+V228E+T252A+L264A; or A20T+G163N+D165S+T252A+L264A (using SEQ ID NO: 2 for numbering).
33. The composition of any of paragraphs 26-32, comprising substitutions corresponding to: L7F+N8K+Q210E+L227G+T252A+L264A; 8D+101K+S105G+R108Q+R179E+G212E; N8D+R209Q+Q210E+T244N+N248K+Q249E;V60E+S83T+T91A+H198S+T252A+L264P; or V60M+T91A+Q210E+V228L+T252A+L264Y (using SEQ ID NO: 2 for numbering).
34. The composition of any of paragraphs 26-33, comprising substitutions corresponding to: V60K+S83T+T91A+H198I+V228L+T252A+L264P; or V60M+A157V+Q210E+L227V+V228A+T252A+L264V (using SEQ ID NO: 2 for numbering).
35. The composition of any of paragraphs 26-34, comprising substitutions corresponding to: D1E+A4Q+L7F+K11N+S37T+A46K+A133R+V142F+T170S+V2021+Q210E+L227G (using SEQ ID NO: 2 for numbering).
36. The composition of any of proceeding paragraphs, wherein the additional enzyme is a protease, a hexosaminidases, a DNase, an amylase, a cellulase or the combinations thereof, e.g., a combination of a protease and a DNase and/or a hexosaminidases, or a combination of a protease and an amylase.
37. The composition of any of proceeding paragraphs, wherein the composition comprises probiotic spores, and preferably the probiotic spores are of Bacillus origin.
38. The composition of any of proceeding paragraphs, wherein the composition is formulated as a liquid, a gel, a paste, a bar; a powder, a tablet, a pouch having one or more compartments, a single or a multi-compartment unit dose form, or a spray form.
39. The composition of any of proceeding composition paragraphs, wherein said composition is hand dish wash detergent, a professional cleaning detergent, a medical cleaning detergent or a hard surface cleanser.
40. A cleaning composition comprising:
(a) 1 to 60 wt %, e.g., 3-40 wt %, 5-25 wt %, of a surfactant system;
(b) 1 to 40 wt %, e.g., 3-20 wt %, 5-10 wt %, of a solvent, wherein the solvent is selected from the group consisting of a lower alkanol, a benzyl alcohol, a lower alkyl ether, a glycol, an aryl glycol ether, a lower alkyl glycol ether, a glycerol ketal, an ester, a hydrocarbon/ester blend, a dibasic ester, alcohol ethoxylate such a as a linear alcohol ethoxylate, a branched alcohol or oxo alcohol ethoxylate, and combinations thereof; and optionally
(c) 0.001 to 10 wt % of a lipase, preferably said lipase is as defined in any of proceeding composition paragraphs 14-35.
41. The cleaning composition of paragraph 40, wherein the surfactant system comprises one or more surfactants selected from linear alkylbenzenesulfonic acid (LAS), alcohol ethersulfate (AEOS), alkyl sulfate (AS), in particular sodium lauryl sulfate (SLS) and sodium laureth sulfate (SLES), and alcohol ethoxylate (AEO), in particular linear alcohol (C₁₂₋₁₅) ethoxylate.
42. The cleaning composition of paragraphs 40-41, wherein the solvent is present at a level of 2-12 wt %, such as 2.1-10 wt %, 2.2-10 wt %, 2.4-8 wt %, 2.6-8 wt %, 2.8-5 wt %, 3-6 wt %, 4-6 wt %, based on the total weight of the composition, and wherein the solvent comprises: at least a lower alkyl glycol ether, e.g., ethylene glycol monohexyl ether, or e.g., a mixture of ethylene glycol monohexyl ether and dipropylene glycol monobutyl ether; or at least an alcohol ethoxylate e.g., a linear alcohol ethoxylate, a branched alcohol, and/or oxo alcohol ethoxylate,
43. The composition of any of proceeding composition paragraphs, wherein said composition is a spray type detergent and has a pH of below 11, e.g. in the range of 6-9.5, in the range of 6.5-9, in the range of 6.8-8.5, or in the range of 7-8, more preferably have a neutral pH of 6.5-7.5, such as pH 7.0, 7.1 or 7.
44. The composition of any of proceeding composition paragraphs, wherein said composition is formulated as a liquid form and has a pH of below 11, e.g. in the range of 6-9.5, in the range of 6.5-9, in the range of 6.8-8.5, or in the range of 7-8, more preferably have a neutral pH of 6.5-7.5, such as pH 7.0, 7.1 or 7.
45. A method for cleaning an item, comprising contacting the surface with a cleaning composition of any one of proceeding paragraphs, preferably the item is a surface e.g., a hard surface, or a textile.
46. The method of paragraph 45, wherein said contacting is performed by spraying, wiping, pouring, soaking and/or mopping the surface with said hard surface cleaning composition.
47. The method of paragraph 45 or 46, wherein the hard surface is selected from a surface of a steel, a rubber, a plastic, a glass, a ceramic, melamine, wood, coated surfaces, cement countertops, kitchen countertops, and surfaces of a medical device.
48. The method of any of paragraphs 45-47, wherein the lipase is dosed at a concentration of at least 0.0001 mg of enzyme protein per gram of said hard surface cleaning composition, e.g., at least 0.001 mg of enzyme protein, at least 0.006 mg of enzyme protein, at least 0.008 mg of enzyme protein, at least 0.01 mg of enzyme protein, at least 0.1 mg of enzyme protein, at least 0.5 mg of enzyme protein, at least 1 mg of enzyme protein, at least 2 mg of enzyme protein, at least 5 mg of enzyme protein, at least 10 mg of enzyme protein, or at least 20 mg of enzyme protein.
49. A ready-to-use item for cleaning a surface, e.g., a hard surface, comprising any of proceeding cleaning composition.
50. The item of paragraph 49, wherein the item is a wipe or a sponge.
51. The item of paragraph 49 or 50, wherein the item is saturated with any of proceeding hard surface cleaning composition.
   The invention is further illustrated in the following non-limiting examples.

### EXAMPLES

Assay 1: Lipase Activity Determined by p-Nitrophenyl (pNP) Assay

The hydrolytic activity of lipases may be determined by a kinetic assay using p-nitrophenyl acyl esters as substrate. A 100 mM stock solution in DMSO for each of the substrates p-nitrophenyl butyrate (C4), p-nitrophenyl caproate (C6), p-nitrophenyl caprate (C10), p-nitrophenyl laurate (C12) and p-nitrophenyl palmitate (C16) (all from Sigma-Aldrich Danmark A/S, Kirkebjerg Allé 84, 2605 Brøndby; Cat. no.: C3:N-9876, C6: N-0502, C10: N-0252, C12: N-2002, C16: N-2752) is diluted to a final concentration of 1 mM 25 mM in the assay buffer (50 mM Tris; pH 7.7; 0.4% Triton X-100). The lipase in 50 mM Hepes; pH 8.0; 10 ppm Triton X-100; +/−20 mM CaCl₂ are added to the substrate solution in the following final protein concentrations: 0.01 mg/ml; 5×10⁻³ mg/ml; 2.5×10⁻⁴ mg/ml; and 1.25×10⁻⁴ mg/mi in 96-well NUNC plates (Cat. No. 260836, Kamstrupvej 90, DK-4000, Roskilde). Release of p-nitrophenol by hydrolysis of a p-nitrophenyl acyl may be monitored at 405 nm for 5 minutes in 10 second intervals on a Spectra max 190 (Molecular Devices GmbH, Bismarckring 39, 88400 Biberach an der Riss, GERMANY).

### Material and Methods

Lipase A: Thermomyces lanuginosus lipase (TLL) shown in SEQ ID NO: 1 with the following substitutions:

$$E 1 C + D 27 R + G 38 A + F 51 V + D 96 E + K 98 I + D 111 A + G 163 K + H 198 S + Y 220 F + T 231 R + N 233 C + D 254 S + P 256 T$$

Solvent A: Diethylene glycol monobutyl ether (BuCb) (available Dow chemical)
Solvent B: Glycol Ether (EPH) (available from Usolf)
Model detergent M1: 2 wt % of fatty alcohol polyoxyethylene ether-9 (AEO-9). The pH of the model detergent was adjusted to about 11 or 8.5 by using monoethanolamine.
Commercial detergent: DAWN Powerwash spray detergent (available from P&G) was used. This detergent contains about 3 wt % solvent of dipropylene glycol butyl ether (DPGBE).
Model detergent V1: 4 wt % of sodium laureth sulfate, 1 wt % of alkyldimethylamine oxide (OA-2), 2.84 wt % of ethylene glycol monohexyl ether (C6EO1), 2.78 wt % of DPGBE, 3.2 wt % PPG400. Adjust to 100% with water.
Model detergent V2: 4 wt % of sodium laureth sulfate, 1 wt % of alkyldimethylamine oxide (OA-2), 2.84 wt % of ethylene glycol monohexyl ether (C6EO1), 2.78 wt % of DPGBE, 3.2 wt % fatty alcohol polyoxyethylene ether-9 (AEO-9). Adjust to 100% with water.

### Example 1

### Soil Removal Test in the Model Detergent for Hard Surface Use

Six different oils were mixed together in the amount shown in below table 2 to prepare an oil mixture. A stainless-steel card was weighted to get an initial wight (marked as M0). About 0.3 g of the oil mixture was spread onto the stainless-steel card evenly. Afterwards the card soiled with the oil mixture was aged by 120° C. ovens for 1 hour and was kept dry before use.

TABLE 2
Components of oil mixture

| Name | Weight (g) |
|---|---|
| Soybean oil | 64.0 |
| No ammonia caramel pigment | 8.0 |
| Wheat-flour | 12.0 |
| Lard | 8.0 |
| Tallow | 8.0 |
| Glycerin monostearate | 2.4 |

The soiled card was weighted to get a before-wash weight (marked as M1) and was then soaked in a beaker containing different detergent formulations (see table 3 below) for 15 min or 7.5 min at 35° C. Afterwards, the card was taken out from the beaker and dried under room temperature. The dried card was weighted to get an after-wash weight (marked as M2).

The wash performance (or the soil removal rate) of different formulation was calculated as:

$$( M 1 - M 2 ) / ( M 1 - M 0 ) \times 100 \%$$

Three repetitions were carried out for each formulation and the averaged soil removal rate was summarized in below table 3.

The improvement of soil removal rate (ISR) by addition of lipase and/or solvent was calculated as:

Soil removal rate of model detergent with lipase and/or solvent−Soil removal rate of model detergent only

TABLE 3
Soil removal rate by different hard surface detergent
formulations with or without lipase A

| | | Soak Time | Soil Removal | |
|---|---|---|---|---|
| Formulation | | (min) | Rate | ISR |
| 1 | Model detergent, pH 11 | 15 | 62.7% | / |
| 2 | Model detergent, pH 8.5 | 15 | 52.7% | / |
| 3 | Model detergent + 1 wt % | 15 | 55.9% | 3.2% |
| | EPH, PH 8.5 | | | |
| 4 | Model detergent + 1% wt % | 15 | 58.0% | 5.2% |
| | (about 380 ppm) lipase, PH | | | |
| | 8.5 | | | |
| 5 | Model + 1 wt % EPH + 1% | 15 | 69.3% | 16.6% |
| | wt % lipase, PH 8.5 | | | |
| 6 | Model detergent + 1 wt % | 15 | 60.8% | 8.1% |
| | Bucb, PH 8.5 | | | |
| 7 | Model detergent + 1% wt % | 15 | 59.4% | 6.7% |
| | lipase, PH 8.5 | | | |
| 8 | Model detergent + 1 wt % | 15 | 72.2% | 19.5% |
| | Bucb + 1% wt % lipase, PH 8.5 | | | |
| 9 | Model detergent + 1 wt % | 7.5 | 52.4% | / |
| | EPH + 1% wt % lipase, pH 8.5 | | | |
| 10 | Model detergent + 1 wt % | 7.5 | 62.5% | / |
| | Bucb + 1% wt % lipase, pH 8.5 | | | |

As can be seen from the above table 2, the soil removal rate decreased when the pH of the detergent decreased from pH 11 to pH 8.5 (formulation 2 vs formulation 1). Under the reduced pH, adding lipase or solvent to the hard surface model detergent compensated part of the loss in wash performance (formulation 3 or 4 vs formulation 2; formulation 6 or 7 vs formulation 2). Surprisingly, when adding both lipase and solvent together to the model detergent, the soil removal rate was greatly improved and even exceeded that of the original high pH model detergent.

In addition, by comparing formulation 9 or 10 with formulation 2, it is clear that inclusion of both lipase and solvent together into a hard surface detergent can deliver similar wash performance in a much shorter time (50% shortened), or in other words can improve the wash efficiency of the detergent.

The enhanced cleaning effect due to the synergy effect of lipase and solvent allows a hard surface cleaning detergent to be formulated as a low or a mild pH (e.g., pH 9 or even lower) detergent without compromising the wash performance as compared to the conventional high pH formulation.

### Example 2

### Confirmation of Enhanced Cleaning Effect in a Commercial Detergent

DAWN Powerwash spray detergent (available from P&G), which contains solvents such as dipropylene glycol butyl ether and hexyl ethoxylate. 50 ppm of Lipase A was added to the detergent.

### Example 2a: Test on Homemade Greasy Stain

Home-made Greasy stain preparation:
1) Melted beef fat in water bath at 70° C.
2) Use pipette to take out 40-100 μL of melted beef fat to apply on 6-cm diameter circle on a tile.
3) Use figures to spread stain evenly on the surface of the tile.
4) Wait around 2-4 hours prior wash test.

### Wash Test:

1) Prepare spray detergent containing 50 ppm lipase.
2) Spray the detergent with/without lipase once to the stain (1 full trigger spray).
3) Leave 15 mins or 1 min (see below table 4) at room temperature and then rinse gently under running water for about 20 sec.
4) Prepare 0.3% Oil Red O (available from Sigma) solution with 75% ethanol.
5) Dip washed tile to 0.3% Oil Red O solution to visualize the residual greasy stain. More residual stain there are, more reddish the tile will appear.
6) Dry the tile at room temperature.
7) Measure R460 nm of the dried tile with DigiEye (Model no: DigiEye Colour Measurement and Imaging System, Serial No. DE0114501). Higher R460 nm value represents less reddish of the tile and hence higher soil removal level.
8) 9 repetitions were carried out and the averaged R460 nm value was listed in below table 4.

TABLE 4
Enhanced cleaning effect was confirmed in
a solvent containing commercial detergent

| Conditions | | R460 nm |
|---|---|---|
| 1 | Original DAWN, pH 10.9, (15 min soaking) | 22.7 |
| 2 | pH 7, 80% DAWN (i.e., 20% reduction of detergent | 26.1 |
| | amount), (1 min soaking) | |
| 3 | pH 7, 80% DAWN + 50 ppm lipase, (1 min soaking) | 53.7 |

Results showed in Table 4 confirmed the aforementioned enhanced cleaning effect (improved soil removal level and improved wash efficiency) and in addition also suggested that by using both lipase and solvent, the amount of detergent can be reduced (e.g., 20% reduction) while still provide an even better soil removal performance (condition 3 vs 1).

### Example 2b: Test with Stubborn Technical Stain DM-90

### Wash Test:

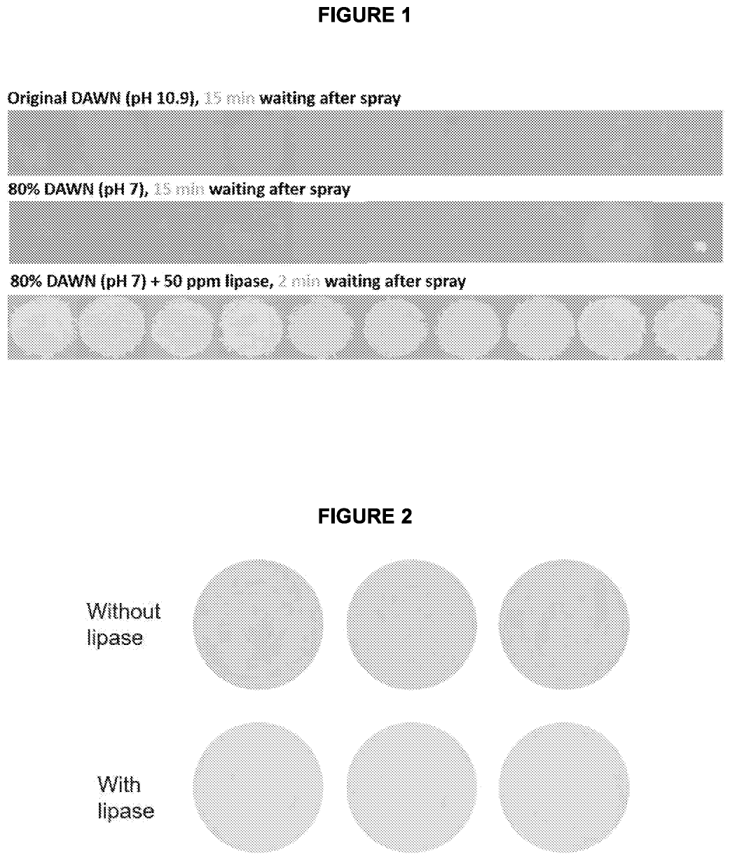

1) Prepare spray detergent containing 50 ppm lipase.
2) Spray the detergent with/without lipase three times to the DM-90 stain tile (available from CFT, Center For Testmaterials).
3) Leave 15 mins or 2 min (see below table 5) at room temperature and then rinse gently under running water for about 20 sec.
4) Dry the tile at room temperature.
5) Measure R460 nm of the dried tile with DigiEye (Model no: DigiEye Colour Measurement and Imaging System, Serial No. DE0114501). Higher R460 nm value represents higher soil removal level or soil removal performance.
6) 10 repetitions were carried out and the averaged R460 nm value was listed in below table 5. The pictures of the 10 repetitions were shown in FIG. 1. From FIG. 1, it is clear that under condition 1 or 2, the whole plates are quite reddish, indicating that most stains are still remained. On the contrary, under condition 3, in which the detergent containing both solvent and lipase, the reddish area is replaced by a whitish/greyish circle (which is the area contacted by detergent), indicating that the stains are removed.

TABLE 5
Enhanced cleaning effect was confirmed in
a solvent containing commercial detergent

| | |
|---|---|
| Conditions | R460 nm |

| | | |
|---|---|---|
| 1 | Original DAWN, pH 10.9, soaking for 15 min | 51.4 |
| 2 | 80% DAWN (i.e., 20% reduction of detergent amount), | 48.6 |
| | pH 7, soaking for 15 min | |
| 3 | 80% DAWN + 0.13 wt % lipase, pH 7, soaking for 2 min | 64.9 |

Results showed in Table 5 confirmed the aforementioned enhanced cleaning effect and in addition also suggested that by using both lipase and solvent, the amount of detergent can be reduced (e.g., 20% reduction) while still provide an even better soil removal performance (condition 3 vs 1) on stubborn technical stain.

### Example 3

### Greasy Stain Removal Test in Dish Wash Model Detergents

### a) Preparation of Beef Tallow Stain

1) Melt beef tallow in water bath at 70° C.
2) Use pipette to take out 40 μl of melted beef fat to apply on 6-cm diameter circle on a melamine tile.
3) Use figures to spread stain evenly on the surface of the tile.
4) Wait around 2-4 hours prior wash test.

### b) Preparation of Mixed Greasy Stain

5) Melt mixed greasy stain (hotpot base seasoning which contains beef tallow: salad oil is roughly 3:2) in water bath at 70° C.
6) Use pipette to take out 40 μl of the melted mixed greasy stain to apply on 6-cm diameter circle on a melamine tile.
7) Use figures to spread stain evenly on the surface of the tile.
8) Wait around 2-4 hours prior wash test.

### Wash Performance Test

1) Prepare model detergents V1 and V2 with or without 77 ppm lipase A. The pH of the detergent was adjusted to pH9, 10 or 10.5 by using sodium hydroxide.
2) Spray the detergent with/without lipase A for 3 times on each stain.
3) Leave 15 mins at room temperature and then rinse gently under running water for about 10 sec.
4) Dry the tiles with mixed grease stain at room temperature. For the tile stained with beef tallow, carry out below steps 5) and 6) to visualize the residual stain before measurement.
5) Prepare 0.3% Oil Red O (available from Sigma) solution with 75% ethanol.
6) Dip washed beef tallow tile to 0.3% Oil Red O solution to visualize the residual greasy stain and dry the tiles at room temperature. More residual stain there are, more reddish the tile will appear.
7) Measure R460 nm of the dried tiles with DigiEye (Model no: DigiEye Colour Measurement and Imaging System, Serial No. DE0114501). Higher R460 nm value represents higher soil removal level, or cleaner tile.
8) 3 repetitions were carried out and the averaged R460 nm value was listed in below tables 6 to 8.

TABLE 6
Lipase performance in model detergent V1

| | | R460 nm | |
|---|---|---|---|
| Conditions | | Beef tallow | Mixed grease |
| 1 | Model detergent V1, pH 8, soaking for 15 min | 12.4 | 53.5 |
| 2 | Model detergent V1 + 77 ppm lipase A, pH 8, | 37.7 | 80.7 |
| | soaking for 15 min | | |

TABLE 7
Lipase performance in model detergent V2 on mixed greasy stains

| | | R460 nm |
|---|---|---|
| Conditions | | Mixed grease stain |
| 1 | Model detergent V2, pH 8, soaking for 15 min | 54.8 |
| 2 | Model detergent V2 + 77 ppm lipase A, pH 8, | 85.6 |
| | soaking for 15 min | |

TABLE 8
Lipase performance in model detergent V2 on mixed greasy stains

| | R460 nm |
|---|---|
| Conditions | Mixed grease stain |

| | | |
|---|---|---|
| 1 | Model detergent V2, pH 9, soaking for 15 min | 59.4 |
| 2 | Model detergent V2 + 77 ppm lipase A, pH 9, | 82.6 |
| | soaking for 15 min | |
| 3 | Model detergent V2, pH 10, soaking for 15 min | 56.0 |
| 4 | Model detergent V2 + 77 ppm lipase A, pH 10, | 77.7 |
| | soaking for 15 min | |
| 5 | Model detergent V2, pH 10.5, soaking for 15 min | 56.7 |
| 6 | Model detergent V2 + 77 ppm lipase A, pH 10.5, | 81.0 |
| | soaking for 15 min | |

From Tables 6, Table 7, Table 8 and FIG. 2, it is clear that model detergent V1 and V2 can shows show significant and consistent lipase performance on greasy stains. The significant wash effect of lipase is also perceivable by naked eyes, see FIG. 2.

### Example 4

### Greasy Stain Removal Test on Fabric

### Preparation of Mixed Greasy Stain

1) Melt mixed greasy stain (hotpot base seasoning which contains beef tallow: salad oil is roughly 3:2) in water bath at 70° C.
2) Use pipette to take out 30 μl of the melted mixed greasy stain to apply on PCN01 fabric, use fingers to spread stain evenly on the surface of the fabric; the final circle of the stain was around 2.8 cm.
3) Wait overnight prior wash test.

### Wash Performance Test

1) Prepare model detergents V2 with or without 77 ppm lipase A.
2) Spray the detergent with/without lipase A for 3 times on each stain.
3) Leave 15 mins at room temperature and then rinse under running water for about 10 sec.
4) Dry the fabric thoroughly at room temperature.
5) Measure R460 nm of the dried fabric with Datacolor 800V (Serial No. 8814595). Higher R460 nm value represents higher soil removal level, or cleaner fabric.
6) 3 repetitions were carried out and the averaged R460 nm value of the three repetitions was listed in below table 9.

TABLE 9
Synergy effect on textiles

| | R460 nm |
|---|---|
| Detergent only, 15 min | 22.1 |
| Detergent + 77 ppm lipase A, 15 min | 35.8 |

From Tables 9, the detergent containing the solvent of the present invention can boost lipase performance on greasy stains that are present on textiles.

1. A hard surface cleaning composition comprising:
(a) 0.1 to 60 wt % of a surfactant system;
(b) 0.1 to 40 wt % of a solvent, wherein the solvent is selected from the group consisting of a lower alkanol, a benzyl alcohol, a lower alkyl ether, a glycol, an aryl glycol ether, a lower alkyl glycol ether, a glycerol ketal, an ester, a hydrocarbon/ester blend, a dibasic ester, alcohol ethoxylate such as a linear alcohol ethoxylate, a branched alcohol or oxo alcohol ethoxylate, and combinations thereof; and optionally
(c) 0.001 to 10 wt % of a lipase.

2. The composition of claim 1, wherein the surfactant system comprises at least one surfactant selected from the group consisting of nonionic surfactants, anionic surfactants, cationic surfactants, ampholytic surfactants, zwitterionic surfactants, semi-polar nonionic surfactants, and mixtures thereof.

3. The composition of claim 1, wherein the solvent is selected from ethanol, propanol, isopropanol and butanol, isobutanol, ethylene glycol, diethylene glycol, triethylene glycol, propylene glycol, dipropylene glycol, mixed ethylene-propylene glycol ethers, ethylene glycol phenyl ether, propylene glycol phenyl ether, propylene glycol methyl ether, propylene glycol propyl ether, dipropylene glycol methyl ether, tripropylene glycol butyl ether, tripropylene glycol methyl ether, ethylene glycol butyl ether, diethylene glycol methyl ether, diethylene glycol butyl ether, ethylene glycol dimethyl ether, ethylene glycol propyl ether, diethylene glycol ethyl ether, triethylene glycol methyl ether, triethylene glycol ethyl ether, triethylene glycol butyl ether, ethylene glycol monobutyl ether, diethylene glycol monobutyl ether, triethylene glycol monobutyl ether, dipropylene glycol mono butyl ether, diethylene glycol monobutyl ether, hexyl ethoxylate (e.g., ethylene glycol monohexyl ether), 2-ethoxyethylalcohol, 2-(2-ethoxyethoxy)ethanol, triethylene glycol monoethyl ether, 2-(hexyloxy)ethanol, 2-(2-hexyloxyethoxy)ethanol, phenoxyethanol, and combination thereof, e.g. a combination of ethanol and diethylene glycol monobutyl ether, glycol ether, propylene glycol or dipropylene glycol.

4. The composition of claim 1, wherein the lipase is
i) a lipase having at least 60%, at least 65%, at least 70%, at least 75%, at least 80%, at least 85%, at least 90%, at least 95% identity, at least 96%, at least 97%, at least 98%, or at least 99%, or 100% sequence identity to SEQ ID NO: 1, 2, 3, 4. 5, 6 or 7;
ii) a variant having lipase activity having at least 60%, at least 70%, at least 80%, at least 90%, at least 91%, at least 92%, at least 93%, at least 94%, at least 95%, at least 96%, at least 97%, at least 98%, or at least 99%, but less than 100% sequence identity to the lipase shown as SEQ ID NO: 1, 2, 3, 4. 5, 6 or 7;
iii) a fragment of the lipase in (i) or (ii) having lipase activity.

5. The composition of claim 4, wherein the lipase is a variant comprising substitutions at positions corresponding to T231R+N233R and optionally at least one or more of D96E, D111A, D254S, G163K, P256T, G91T and G38A of SEQ ID NO: 1.

6. The composition of claim 1, wherein the lipase is a variant of a parent lipase, wherein the variant has lipase activity and has at least 60%, at least 65%, at least 70%, at least 75%, at least 80%, at least 85%, at least 90%, at least 95%, at least 96%, at least 97%, at least 98%, at least 99%, but less than 100% sequence identity with SEQ ID NO: 1, and comprises substitutions at positions corresponding to T231R+N233R and at least one or more of D96E, D111A, D254S, G163K, P256T, G91T and G38A of SEQ ID NO: 1 selected from the group of:
$$\begin{matrix}a . & D 96 E + T 231 R + N 233 R ; \\ b . & N 33 Q + D 96 E + T 231 R + N 233 R ; \\ c . & N 33 Q + D 111 A + T 231 R + N 233 R ; \\ d . & N 33 Q + T 231 R + N 233 R + P 256 T ; \\ e . & N 33 Q + G 38 A + G 91 T + G 163 K + T 231 R + N 233 R + D 254 S ; \\ f . & N 33 Q + G 38 A + G 91 T + D 96 E + D 111 A + G 163 K + T 231 R + N 233 R + D 254 S + P 256 T ; \\ g . & D 27 R + N 33 Q + G 38 A + D 96 E + D 111 A + G 163 K + T 231 R + N 233 R + D 254 S + P 256 T ; \\ h . & D 27 R + N 33 Q + G 38 A + G 91 T + D 96 E + D 111 A + G 163 K + T 231 R + N 233 R + P 256 T ; \\ i . & D 27 R + N 33 Q + G 38 A + G 91 T + D 96 E + D 111 A + G 163 K + T 231 R + N 233 R + D 254 S ; \\ j . & D 27 R + G 38 A + G 91 T + D 96 E + D 111 A + G 163 K + T 231 R + N 233 R + D 254 S + P 256 T ; \\ k . & D 96 E + T 231 R + N 233 R + D 254 S ; \\ l . & T 231 R + N 233 R + D 254 S + P 256 T ; \\ m . & G 163 K + T 231 R + N 233 R + D 254 S ; \\ n . & D 27 R + N 33 Q + G 38 A + G 91 T + D 96 E + G 163 K + T 231 R + N 233 R + D 254 S + P 256 T ; \\ o . & D 27 R + G 91 T + D 96 E + D 111 A + G 163 K + T 231 R + N 233 R + D 254 S + P 256 T ; \\ p . & D 96 E + G 163 K + T 231 R + N 233 R + D 254 S ; \\ q . & D 27 R + G 163 K + T 231 R + N 233 R + D 254 S ; \\ r . & D 27 R + G 38 A + G 91 T + D 96 E + D 111 A + G 163 K + T 231 R + N 233 R + D 254 S ; \\ s . & D 27 R + G 38 A + G 91 T + D 96 E + G 163 K + T 231 R + N 233 R + D 254 S + P 256 T ; \\ t . & D 27 R + G 38 A + D 96 E + D 111 A + G 163 K + T 231 R + N 233 R + D 254 S + P 256 T ; \\ u . & D 27 R + D 96 E + G 163 K + T 231 R + N 233 R + D 254 S ; \\ v . & D 27 R + D 96 E + D 111 A + G 163 K + T 231 R + N 233 R + D 254 S + P 256 T ; \\ w . & D 27 R + G 38 A + D 96 E + G 163 K + T 231 R + N 233 R + D 254 S + P 256 T \\ x . & D 111 A + G 163 K + T 231 R + N 233 R + D 254 S + P 256 T ; \\ y . & D 111 A + T 231 R + N 233 R ; \\ z . & D 111 A + T 231 R + N 233 R + D 254 S + P 256 T ; \\ aa . & D 27 R + D 96 E + D 111 A + G 163 K + T 231 R + N 233 R ; \\ bb . & D 27 R + D 96 E + D 111 A + T 231 R + N 233 R ; \\ cc . & D 27 R + N 33 Q + G 38 A + D 96 E + D 111 A + T 231 R + N 233 R + D 254 S + P 256 T ; \\ dd . & D 27 R + G 38 A + D 96 E + D 111 A + G 163 K + E 210 Q + T 231 R + N 233 R + D 254 S + P 256 T ; \\ ee . & D 27 R + T 231 R + N 233 R + D 254 S + P 256 T ; \\ ff . & D 96 E + D 111 A + G 163 K + T 231 R + N 233 R ; \\ gg . & D 96 E + D 111 A + G 163 K + T 231 R + N 233 R + D 254 S + P 256 T ; \\ hh . & D 96 E + D 111 A + G 163 K + T 231 R + N 233 R + P 256 T ; \\ ii . & D 96 E + D 111 A + T 231 R + N 233 R ; \\ jj . & D 96 E + D 111 A + T 231 R + N 233 R + D 254 S ; \\ kk . & D 96 E + D 111 A + T 231 R + N 233 R + D 254 S + P 256 T \\ ll . & D 96 E + D 111 A + T 231 R + N 233 R + P 256 T ; \\ m m . & D 96 E + G 163 K + T 231 R + N 233 R + D 254 S + P 256 T ; \\ nn . & D 96 E + T 231 R + N 233 R + D 254 S + P 256 T ; \\ oo . & D 96 E + T 231 R + N 233 R + P 256 T ; \\ pp . & G 38 A + D 96 E + D 111 A + T 231 R + N 233 R ; \\ qq . & G 38 A + D 96 E + D 111 A + G 163 K + T 231 R + N 233 R + D 254 S + P 256 T ; \\ rr . & G 91 T + D 96 E + D 111 A + T 231 R + N 233 R ; \\ ss . & G 91 T + D 96 E + T 231 R + N 233 R ; \\ tt . & G 91 T + T 231 R + N 233 R + D 254 S + P 256 T ; \\ uu . & N 33 Q + D 96 E + D 111 A + G 163 K + T 231 R + N 233 R + D 254 S + P 256 T ; \\ vv . & T 231 R + N 233 R + D 254 S + P 256 T ; \\ ww . & T 231 R + N 233 R + P 256 T .\end{matrix}$$

7. The composition of claim 1, wherein the lipase variant comprises substitutions E1C+N233C and one or more additional substitutions, wherein numbering is according to SEQ ID NO: 1.

8. The composition of claim 1, wherein the variant has lipase activity and has at least 60%, at least 65%, at least 70%, at least 75%, at least 80%, at least 85%, at least 90%, at least 95%, at least 96%, at least 97%, at least 98%, at least 99%, but less than 100% sequence identity with SEQ ID NO: 1 and comprises or consists of substitutions corresponding to one of the following set of substitutions using SEQ ID NO: 1 for numbering:
$$\begin{matrix}E 1 C + H 198 L + N 233 C \\ E 1 C + H 198 G + N 233 C \\ E 1 C + L 69 V + N 233 C \\ E 1 C + L 69 T + N 233 C \\ E 1 C + L 69 S + N 233 C \\ E 1 C + L 69 H + N 233 C \\ E 1 C + L 69 F + N 233 C \\ E 1 C + L 69 C + N 233 C \\ E 1 C + H 198 Y + N 233 C \\ E 1 C + H 198 T + N 233 C \\ E 1 C + H 198 G + N 233 C \\ E 1 C + L 227 F + N 233 C \\ E 1 C + L 227 R + N 233 C \\ E 1 C + E 210 T + N 233 C \\ E 1 C + E 210 N + N 233 C \\ E 1 C + V 176 M + N 233 C \\ E 1 C + K 98 T + N 233 C \\ E 1 C + K 98 E + N 233 C \\ E 1 C + E 56 S + N 233 C \\ E 1 C + E 56 Q + N 233 C \\ E 1 C + E 56 R + N 233 C \\ E 1 C + F 51 M + N 233 C \\ E 1 C + D 27 R + F 51 Y + N 233 C \\ E 1 C + V 2 I + N 233 C \\ E 1 C + V 2 N + N 233 C \\ E 1 C + V 2 K + N 233 C \\ E 1 C + V 2 A + N 233 C \\ E 1 C + D 96 L + N 233 C \\ E 1 C + L 69 R + N 233 C \\ E 1 C + V 2 Y + N 233 C \\ E 1 C + N 233 C + P 256 T \\ E 1 C + N 233 C + D 254 S \\ E 1 C + T 231 R + N 233 C \\ E 1 C + H 198 S + N 233 C \\ E 1 C + D 111 A + N 233 C \\ E 1 C + D 96 E + N 233 C \\ E 1 C + G 38 A + N 233 C \\ E 1 C + N 33 Q + N 233 C \\ E 1 C + N 33 K + N 233 C \\ E 1 C + E 210 A + N 233 C \\ E 1 C + E 210 Q + N 233 C \\ E 1 C + E 210 R + N 233 C \\ E 1 C + H 198 D + N 233 C \\ E 1 C + K 98 R + N 233 C \\ E 1 C + K 98 V + N 233 C \\ E 1 C + F 51 L + N 233 C \\ E 1 C + F 51 I + N 233 C \\ E 1 C + K 237 C \\ E 1 C + L 227 G + N 233 C \\ E 1 C + E 210 K + N 233 C \\ E 1 C + V 176 L + N 233 C \\ E 1 C + K 98 Q + N 233 C \\ E 1 C + E 56 K + N 233 C \\ E 1 C + L 147 S + N 233 C + D 254 S \\ E 1 C + Y 220 F + N 233 C \\ E 1 C + K 98 I + N 233 C \\ E 1 C + N 233 C \\ E 1 C + D 27 R + F 51 I + E 56 R + K 98 E + T 231 R + N 233 C \\ E 1 C + D 27 R + F 51 I + E 56 R + K 98 E + T 231 R + N 233 C + D 254 S \\ E 1 C + D 27 R + G 38 A + F 51 L + K 98 I + D 111 A + G 163 S + H 198 S + Y 220 F + T 231 R + N 233 C + P 256 T \\ E 1 C + D 27 R + G 38 A + F 51 L + D 96 E + K 98 I + D 111 A + G 163 K + H 198 S + Y 220 F + T 231 R + N 233 C + D 254 S + P 256 T \\ E 1 C + D 27 R + G 38 R + F 51 L + D 96 E + K 98 I + D 111 A + G 163 K + H 198 S + Y 220 F + T 231 R + N 233 C + D 254 S + P 256 T \\ E 1 C + D 27 R + F 51 L + D 96 I + K 98 I + D 111 A + G 163 K + H 198 S + Y 220 F + T 231 R + N 233 C + P 256 T \\ E 1 C + D 27 R + F 51 L + D 96 E + K 98 I + D 111 A + G 163 S + H 198 S + Y 220 F + T 231 R + N 233 C + P 256 T \\ E 1 C + D 27 R + F 51 L + D 96 E + K 98 I + D 111 A + G 163 K + H 198 S + Y 220 F + T 231 R + N 233 C + P 256 T \\ E 1 C + D 27 R + G 38 A + F 51 I + D 96 E + K 98 I + D 111 A + G 163 K + H 198 S + Y 220 F + T 231 R + N 233 C + D 254 S + P 256 T \\ E 1 C + D 27 R + G 38 A + F 51 V + D 96 E + K 98 I + D 111 A + G 163 K + H 198 S + Y 220 F + T 231 R + N 233 C + P 256 T \\ E 1 C + D 27 R + F 51 V + D 96 E + K 98 I + D 111 A + G 163 K + H 198 S + Y 220 F + T 231 R + N 233 C + P 256 T \\ E 1 C + D 27 R + F 51 V + D 96 E + K 98 I + D 111 A + G 163 S + H 198 S + Y 220 F + T 231 R + N 233 C + D 254 S + P 256 T \\ E 1 C + D 27 R + F 51 V + D 96 I + K 98 I + D 111 A + G 163 K + H 198 S + Y 220 F + T 231 R + N 233 C + D 254 S + P 256 T \\ E 1 C + D 27 R + F 51 V + K 98 I + D 111 A + G 163 K + H 198 S + Y 220 F + T 231 R + N 233 C + D 254 S + P 256 T \\ E 1 C + D 27 R + F 51 V + D 96 E + K 98 I + D 111 A + G 163 K + H 198 S + Y 220 F + T 231 R + N 233 C + D 254 S + P 256 T \\ E 1 C + D 27 R + G 38 A + F 51 V + D 96 E + K 98 I + D 111 A + G 163 K + H 198 S + Y 220 F + T 231 R + N 233 C + D 254 S + P 256 T \\ E 1 C + F 51 V + D 96 E + K 98 I + D 111 A + G 163 S + H 198 S + Y 220 F + T 231 R + N 233 C + P 256 T \\ E 1 C + F 51 L + D 96 E + K 98 I + D 111 A + G 163 K + H 198 S + Y 220 F + T 231 R + N 233 C + D 254 S + P 256 T \\ E 1 C + G 38 A + F 51 V + D 96 E + K 98 I + D 111 A + G 163 K + H 198 S + Y 220 F + T 231 R + N 233 C + P 256 T \\ E 1 C + F 51 V + D 96 E + K 98 I + D 111 A + G 163 K + H 198 S + Y 220 F + T 231 R + N 233 C + P 256 T \\ E 1 C + F 51 V + D 96 I + K 98 I + D 111 A + G 163 K + H 198 S + Y 220 F + T 231 R + N 233 C + D 254 S + P 256 T \\ E 1 C + F 51 V + K 98 I + D 111 A + G 163 K + H 19 S + Y 220 F + T 231 R + N 233 C + D 254 S + P 256 T \\ E 1 C + F 51 I + D 96 E + K 98 I + D 111 A + G 163 K + H 198 S + Y 220 F + T 231 R + N 233 C + D 254 S + P 256 T \\ E 1 C + D 27 R + F 51 L + D 96 E + K 98 I + D 111 A + G 163 K + H 198 S + Y 220 F + T 231 R + N 233 C + D 254 S + P 256 T \\ E 1 C + D 27 R + N 33 K + G 38 A + F 51 V + D 96 E + K 98 I + D 111 A + G 163 K + H 198 S + Y 220 F + T 231 R + N 233 C \\ E 1 C + G 38 R + F 51 V + D 96 E + K 98 I + D 111 A + G 163 K + H 198 S + Y 220 F + T 231 R + N 233 C + D 254 S + P 256 T \\ E 1 C + F 51 V + D 96 E + K 98 I + D 111 A + G 163 K + H 198 S + Y 220 F + T 231 R + N 233 C + D 254 S + P 256 T \\ E 1 C + G 38 A + F 51 V + D 96 E + K 98 I + D 111 A + G 163 K + H 198 S + Y 220 F + T 231 R + N 233 C + D 254 S + P 256 T \\ E 1 C + D 27 R + G 38 R + F 51 V + D 96 E + K 98 I + D 111 A + G 163 K + H 198 S + Y 220 F + T 231 R + N 233 C + D 254 S + P 256 T .\end{matrix}$$

9. The composition of claim 1, wherein the lipase is
i) a lipase having at least 60%, at least 65%, at least 70%, at least 75%, at least 80%, at least 85%, at least 90%, at least 95% identity, at least 96%, at least 97%, at least 98%, or at least 99%, or 100% sequence identity to SEQ ID NO: 2;
ii) a variant having lipase activity having at least 60%, at least 70%, at least 80%, at least 90%, at least 91%, at least 92%, at least 93%, at least 94%, at least 95%, at least 96%, at least 97%, at least 98%, or at least 99%, but less than 100% sequence identity to the lipase shown as SEQ ID NO: 2;
iii) a fragment of the lipase in (i) or (ii) having lipase activity.

10. The composition of claim 1, wherein the composition comprises or consists of the amino acid of SEQ ID NO: 2.

11. The composition of claim 16, wherein the additional enzyme is a protease, a hexosaminidases, a DNase, an amylase, a cellulase or the combinations thereof, e.g., a combination of a protease and a DNase and/or a hexosaminidases, or a combination of a protease and an amylase.

12. The composition of claim 1, wherein the composition comprises probiotic spores.

13. The composition of claim 1, wherein said composition is a spray type detergent and has a pH of below 11.

14. A method for cleaning a hard surface, comprising contacting the surface with a hard surface cleaning composition of claim 1.

15. A cleaning composition comprising:
(a) 1 to 60 wt %, e.g., 3-40 wt %, 5-25 wt %, of a surfactant system;
(b) 1 to 40 wt %, e.g., 3-20 wt %, 5-10 wt %, of a solvent, wherein the solvent is selected from the group consisting of a lower alkanol, a benzyl alcohol, a lower alkyl ether, a glycol, an aryl glycol ether, a lower alkyl glycol ether, a glycerol ketal, an ester, a hydrocarbon/ester blend, a dibasic ester, alcohol ethoxylate such as a linear alcohol ethoxylate, a branched alcohol or oxo alcohol ethoxylate, and combinations thereof; and optionally
(c) 0.001 to 10 wt % of a lipase.

16. The composition of claim 1, further comprising an additional enzyme.

17. The composition of claim 12, wherein the probiotic spores are of Bacillus origin.

18. The composition of claim 13, wherein said composition has a pH in the range of 6-9.5.

19. The composition of claim 13, wherein said composition has a pH in the range of 7-8.
